(19) United States
(12) Patent Application Publication
(10) Pub. No.: US 2014/0024028 A1
(43) Pub. Date: Jan. 23, 2014

(54) BRCA DEFICIENCY AND METHODS OF USE

(71) Applicants: Kirsten Timms, Salt Lake City, UT (US); Darl Flake, Salt Lake City, UT (US); Jennifer Potter, Salt Lake City, UT (US); Jerry Lanchbury, Salt Lake City, UT (US)

(72) Inventors: Kirsten Timms, Salt Lake City, UT (US); Darl Flake, Salt Lake City, UT (US); Jennifer Potter, Salt Lake City, UT (US); Jerry Lanchbury, Salt Lake City, UT (US)

(73) Assignee: MYRIAD GENETICS, INC., Salt Lake City, UT (US)

(21) Appl. No.: 13/852,129

(22) Filed: Mar. 28, 2013

Related U.S. Application Data
(63) Continuation of application No. PCT/US11/54369, filed on Sep. 30, 2011.

(51) Int. Cl. C12Q 1/68

(52) U.S. Cl. CPC C12Q 1/6886
USPC 435/6.11; 435/6.12; 435/287.2

(57) ABSTRACT

The invention generally relates to a molecular classification of disease and particularly to methods and compositions for determining BRCA deficiency.

### CROSS-REFERENCE TO RELATED APPLICATIONS

This application is a continuation of International Application No. PCT/US11/054,369, filed Sep. 30, 2011, which claims priority benefit of U.S. Provisional Application No. 61/388,692, filed Oct. 1, 2010. The contents of each of these prior applications are hereby incorporated by reference in their entirety.

### FIELD OF THE INVENTION

The invention generally relates to a molecular classification of disease and particularly to methods and compositions for determining BRCA deficiency.

### TABLES

The instant application was filed with one (1) table (Table 1) under 37 C.F.R. §§1.52(e)(1)(iii) & 1.58(b), submitted electronically as the following text file: “3317-01-1P-2010-10-01-TABLE1-BGJ.txt”; creation date: Oct. 1, 2010; Size: 86,503 bytes. This file and all its contents are incorporated by reference herein in their entirety.

LENGTHY TABLES
The patent application contains a lengthy table section. A copy of the table is available in electronic form from the USPTO web site (http://seqdata.uspto.gov/?pageRequest=docDetail&DocID=US20140024028A1). An electronic copy of the table will also be available from the USPTO upon request and payment of the fee set forth in 37 CFR 1.19(b)(3).

### BACKGROUND OF THE INVENTION

The breast and ovarian cancer susceptibility genes, BRCA1 and BRCA2, were discovered in patients having a family history of breast or ovarian cancer. Miki et al., SCIENCE (1994) 266:66-71. The BRCA genes are tumor suppressors found deficient in a large proportion of solid tumors. For example, a significant proportion of sporadic breast and ovarian cancers harbor somatic BRCA mutations. Due to the critical role of BRCA deficiency in tumor formation and progression, identifying BRCA deficiency can be very important, inter alia, in the individualized clinical management of cancer patients (e.g., chemoselection). Thus, it is desirable to identify new markers and methods for detecting BRCA deficiency.

### SUMMARY OF THE INVENTION

It has been discovered that measuring expression of the BRCA1 and/or BRCA2 (referred to collectively as “BRCA”) genes together with cell-cycle progression (“CCP”) gene expression can effectively identifies tumors with BRCA deficiency. Specifically, we determined that tumors in which BRCA and CCP expression are anti-correlated represent a subgroup of BRCA deficient tumors. This subgroup is generally characterized by BRCA hypermethylation. Thus the invention generally provides compositions and methods for determining BRCA status.

In one aspect the invention provides a method for determining gene expression comprising measuring the expression of BRCA1 and/or BRCA2 (BRCA expression) in a sample and measuring the expression of a panel of CCP genes in the sample. Some embodiments further comprise determining whether BRCA expression is correlated to CCP expression. Some embodiments further comprise analyzing methylation in BRCA1 and/or BRCA2 in the sample.

As mentioned above, anti-correlation between BRCA and CCP expression is correlated with BRCA deficiency. Thus another aspect of the invention provides a method for determining whether a sample is BRCA deficient comprising measuring the expression of BRCA1 and/or BRCA2 (BRCA expression) in said sample and measuring the expression of a panel of CCP genes in the sample. Some embodiments further comprise determining whether BRCA expression is correlated to CCP expression. In some embodiments, anti-correlation between BRCA and CCP expression indicates the sample is BRCA deficient. In some embodiments anti-correlation between BRCA and CCP expression indicates the sample has BRCA hypermethylation. Some embodiments further comprise analyzing methylation in BRCA1 and/or BRCA2 in the sample.

In some embodiments the panel of CCP genes comprises at least two (or five, or six, or ten, or 15) CCP genes from any of Tables 1 to 5 or Panels A to G. In some embodiments the panel of CCP genes comprises the genes in any of Tables 1 to 5 or Panels A to G.

In some embodiments, determining the expression of a panel of genes comprising CCP genes involves determining the expression of a plurality of test genes comprising at least 4, 6, 8, 10, 15 or more CCP genes and deriving a test value from the determined expression, wherein the CCP genes are weighted to contribute at least 50%, at least 75% or at least 85% of the test value. Thus, in some embodiments, the invention provides a method for determining whether a sample is BRCA deficient comprising (1) determining in a sample from a patient (a) the expression of BRCA1 and/or BRCA2, and (b) the expression of a panel of genes including at least 4 or at least 8 cell-cycle genes; (2) providing a test value by (a) weighting the determined expression of each of a plurality of test genes selected from the panel of genes with a predefined coefficient, and (b) combining the weighted expression to provide the test value, wherein the cell-cycle genes are weighted to contribute at least 50%, at least 75% or at least 85% of the test value; and (3) comparing the test value to the expression of BRCA 1 and/or BRCA2 to determine whether these are correlated or anti-correlated. In some embodiments the method further comprises (4) correlating an anti-correlation between the test value and BRCA1 and/or BRCA2 expression to BRCA deficiency.

BRCA deficiency is associated with various characteristics in tumors. Thus in one aspect the invention provides a method of classifying a cancer comprising measuring the expression of BRCA1 and/or BRCA2 (BRCA expression) in said sample and measuring the expression of two or more CCP genes in the sample. Some embodiments further comprise determining whether BRCA expression is correlated to CCP expression. In some embodiments, anti-correlation between BRCA and CCP expression indicates any one of the following: greater likelihood of survival (e.g., progression-free survival, overall survival, etc.), greater likelihood of response to DNA damaging agents (e.g., platinum chemotherapy drugs, etc.), greater likelihood of response to drugs targeting the poly (ADP-ribose) polymerase (PARP) pathway, etc. Some embodiments further comprise determining whether BRCA1 and/or BRCA2 is hypermethylated.

In some embodiments gene expression is determined using any of the following techniques: quantitative PCR™ (e.g., TaqMan™), microarray hybridization analysis, quantitative sequencing, etc. In some embodiments methylation is analyzed using any of the following techniques: Southern blotting, single nucleotide primer extension, methylation-specific polymerase chain reaction (MSPCR), restriction landmark genomic scanning for methylation (RLGS-M) and CpG island microarray, single nucleotide primer extension (SNuPE), combined bisulfite restriction analysis (COBRA), etc.

In another aspect the invention provides systems related to the above methods of the invention. In one embodiment the invention provides a system for determining gene expression in a tumor sample, comprising: (1) a sample analyzer for determining the expression levels of BRCA1 and/or BRCA2 and a panel of genes comprising at least two CCP genes in a sample, wherein the sample analyzer contains the sample, mRNA from the sample and expressed from the panel of genes, or cDNA synthesized from said mRNA; (2) a first computer program for (a) receiving gene expression data on BRCA1 and/or BRCA2, (b) receiving gene expression data on at least two test genes selected from the panel of genes, (c) weighting the determined expression of each of the test genes with a predefined coefficient, and (d) combining the weighted expression to provide a CCP test value representing the expression level of the panel of genes.

In some embodiments the above system further comprises a computer program for comparing the expression of BRCA1 and/or BRCA2 to the CCP test value, wherein high expression of BRCA1 and/or BRCA2 coupled with a high CCP test value indicates BRCA and CCP expression are correlated, wherein low expression of BRCA1 and/or BRCA2 coupled with a low CCP test value indicates BRCA and CCP expression are correlated, wherein high expression of BRCA1 and/or BRCA2 coupled with a low CCP test value indicates BRCA and CCP expression are anti-correlated, and wherein low expression of BRCA1 and/or BRCA2 coupled with a high CCP test value indicates BRCA and CCP expression are anti-correlated.

In some embodiments the above system further comprises a computer program for receiving data on the correlation between BRCA expression and CCP expression in a patient sample and concluding that the sample is BRCA deficient if BRCA expression and CCP expression are anti-correlated in the sample. In some embodiments the system comprises a sample analyzer for determining the methylation status of BRCA1 and/or BRCA2.

In yet another aspect the invention provides a kit for practicing the methods and for use in the systems of the present invention. The kit may include a carrier for the various components of the kit. The carrier can be a container or support, in the form of, e.g., bag, box, tube, rack, and is optionally compartmentalized. The carrier may define an enclosed confinement for safety purposes during shipment and storage.

The kit includes various components useful in determining the expression of BRCA1 and/or BRCA2, the expression of at least two CCP genes, and optionally the expression of one or more housekeeping gene markers and/or the methylation status of BRCA1 and/or BRCA2. For example, the kit many include oligonucleotides specifically hybridizing under high stringency to mRNA or cDNA of BRCA1, BRCA2, or the genes in Tables 1 to 5 or Panels A to F. Such oligonucleotides can be used as PCR primers in RT-PCR reactions, or hybridization probes.

Various techniques for determining BRCA status are known to those skilled in the art. In some embodiments the whole genome of one or more cells is determined and the sequence of a BRCA gene found within that genome is analyzed for mutations. In some embodiments a BRCA gene is specifically sequenced, which may include exon sequencing, sequencing of exons along with at least some amount of flanking intronic sequence, or sequencing of the entire genomic region containing the BRCA gene of interest. Copy number analysis may also be used. In some embodiments large rearrangement analysis is used to determine whether large portions of the BRCA gene (or even the entire gene) have been deleted or duplicated. In some embodiments methylation analysis is used to determine BRCA status.

The foregoing and other advantages and features of the invention, and the manner in which the same are accomplished, will become more readily apparent upon consideration of the following detailed description of the invention taken in conjunction with the accompanying examples and drawings, which illustrate preferred and exemplary embodiments.

### BRIEF DESCRIPTION OF THE DRAWINGS

FIG. 1 illustrates how the predictive power of CCP gene signatures varies with the number of CCP genes.

FIG. 2 illustrates the relationship between BRCA1 and cell-cycle expression.

FIG. 3 illustrates embodiments of computer systems of the invention.

FIG. 4 illustrates embodiments of computer-implemented methods of the invention.

FIG. 5 illustrates the correlation between BRCA-CCP expression anti-correlation and BRCA1 hypermethylation.

FIG. 6 shows the pairwise relationships between BRCA1 qPCR assays. Correlations are given in the upper panels.

FIG. 7 a histogram of BRCA1 expression as measured by qPCR.

FIG. 8 shows the relationship between each of the cell-cycle genes and the CCP score.

FIG. 9 shows CCP score and BRCA1 expression.

FIG. 10 shows CCP score and BRCA1 expression separated by ER/PR/HER2 subtype as determined by IHC.

FIG. 11 shows the relationship between BRCA1 promoter methylation and BRCA1 expression.

FIG. 12 shows the relationship between CCP score and BRCA1 expression in samples with BRCA1 methylation data. The size of the points represents the degree of BRCA1 methylation. Each point is colored by tumor subtype as identified by IHC

### DETAILED DESCRIPTION OF THE INVENTION

It has been discovered that measuring BRCA expression together with cell-cycle progression (“CCP”) gene expression can effectively identify tumors with BRCA deficiency (Example 2). Specifically, we determined that tumors in which BRCA and CCP expression are anti-correlated represent a subgroup of BRCA deficient tumors (id.). This subgroup is generally characterized by BRCA hypermethylation (id.). Thus determining BRCA and CCP expression levels can effectively identify BRCA deficient tumors better than BRCA expression alone. Accordingly the invention generally provides compositions and methods for determining BRCA status.

In one aspect the invention provides a method for determining gene expression comprising measuring the expression of BRCA1 and/or BRCA2 (BRCA expression) in a sample and measuring the expression of a panel of CCP genes in the sample. Some embodiments further comprise determining whether BRCA expression is correlated to CCP expression. Some embodiments further comprise analyzing methylation in BRCA1 and/or BRCA2 in the sample.

As mentioned above, anti-correlation between BRCA and CCP expression is correlated with BRCA deficiency. Thus another aspect of the invention provides a method for determining whether a sample is BRCA deficient comprising measuring the expression of BRCA1 and/or BRCA2 (BRCA expression) in said sample and measuring the expression of a panel of CCP genes in the sample. “BRCA deficient” and “BRCA deficiency” mean attenuated cellular activity of BRCA1 and/or BRCA2 protein. This can include deletion of part or all of the BRCA1 and/or BRCA2 gene, lowered transcription and/or stability of BRCA1 and/or BRCA2 mRNA (e.g., as caused by hypermethylation), lowered translation of BRCA1 and/or BRCA2 protein, or mutation(s) in the BRCA1 and/or BRCA2 gene or transcripts leading to a protein with lowered biochemical activity.

“Cell-cycle progression gene” and “CCP gene” herein refer to a gene whose expression level closely tracks the progression of the cell through the cell-cycle. See, e.g., Whitfield et al., MOL. BIOL. CELL (2002) 13:1977-2000. More specifically, CCP genes show periodic increases and decreases in expression that coincide with certain phases of the cell cycle—e.g., STK15 and PLK show peak expression at G2/M. Id. Often CCP genes have clear, recognized cell-cycle related function—e.g., in DNA synthesis or repair, in chromosome condensation, in cell-division, etc. However, some CCP genes have expression levels that track the cell-cycle without having an obvious, direct role in the cell-cycle—e.g., UBE2S encodes a ubiquitin-conjugating enzyme, yet its expression closely tracks the cell-cycle. Thus a CCP gene according to the present invention need not have a recognized role in the cell-cycle. Exemplary CCP genes (and panels of CCP genes) are listed in Tables 1 (Table 1 as shown in U.S. provisional application Ser. No. 61/388,692), 2, 3, 4, and 5 and Panels A, B, C, D, E, and F.

Whether a particular gene is a CCP gene may be determined by any technique known in the art, including that taught in Whitfield et al., MOL. BIOL. CELL (2002) 13:1977-2000. For example, a sample of cells, e.g., HeLa cells, can be synchronized such that they all progress through the different phases of the cell cycle at the same time. Generally this is done by arresting the cells in each phase—e.g., cells may be arrested in S phase by using a double thymidine block or in mitosis with a thymidine-nocodazole block. See, e.g., Whitfield et al., MOL. CELL. BIOL. (2000) 20:4188-4198. RNA is extracted from the cells after arrest in each phase and gene expression is quantitated using any suitable technique—e.g., expression microarray (genome-wide or specific genes of interest), real-time quantitative PCR™ (RTQ-PCR). Finally, statistical analysis (e.g., Fourier Transform) is applied to determine which genes show peak expression during particular cell-cycle phases. Genes may be ranked according to a periodicity score describing how closely the gene's expression tracks the cell-cycle—e.g., a high score indicates a gene very closely tracks the cell cycle. Finally, those genes whose periodicity score exceeds a defined threshold level (see Whitfield et al., MOL. BIOL. CELL (2002) 13:1977-2000) may be designated CCP genes. A large, but not exhaustive, list of nucleic acids associated with CCP genes (e.g., genes, ESTs, cDNA clones, etc.) is given in Table 1. See Whitfield et al., MOL. BIOL. CELL (2002) 13:1977-2000. All of the CCP genes in Table 2 below form a panel of CCP genes (“Panel A”) useful in the methods of the invention.

TABLE 2

| | Entrez | | RefSeq Accession |
|---|---|---|---|
| Gene Symbol | GeneID | ABI Assay ID | Nos. |
| APOBEC3B* | 9582 | Hs00358981_m1 | NM_004900.3 |
| ASF1B* | 55723 | Hs00216780_m1 | NM_018154.2 |
| ASPM* | 259266 | Hs00411505_m1 | NM_018136.4 |
| ATAD2* | 29028 | Hs00204205_m1 | NM_014109.3 |
| BIRC5* | 332 | Hs00153353_m1; | NM_01012271.1; |
| | | Hs03043576_m1 | NM_01012270.1; |
| | | | NM_001168.2 |
| BLM* | 641 | Hs00172060_m1 | NM_000057.2 |
| BUB1 | 699 | Hs00177821_m1 | NM_004336.3 |
| BUB1B* | 701 | Hs01084828_m1 | NM_001211.5 |
| C12orf48* | 55010 | Hs00215575_m1 | NM_017915.2 |
| C18orf24* | 220134 | Hs00536843_m1 | NM_145060.3; |
| | | | NM_001039535.2 |
| C1orf135* | 79000 | Hs00225211_m1 | NM_024037.1 |
| C21orf45* | 54069 | Hs00219050_m1 | NM_018944.2 |
| CCDC99* | 54908 | Hs00215019_m1 | NM_017785.4 |
| CCNA2* | 890 | Hs00153138_m1 | NM_001237.3 |
| CCNB1* | 891 | Hs00259126_m1 | NM_031966.2 |
| CCNB2* | 9133 | Hs00270424_m1 | NM_004701.2 |
| CCNE1* | 898 | Hs01026536_m1 | NM_001238.1; |
| | | | NM_057182.1 |
| CDC2* | 983 | Hs00364293_m1 | NM_033379.3; |
| | | | NM_001130829.1; |
| | | | NM_001786.3 |
| CDC20* | 991 | Hs03004916_g1 | NM_001255.2 |
| CDC45L* | 8318 | Hs00185895_m1 | NM_003504.3 |
| CDC6* | 990 | Hs00154374_m1 | NM_001254.3 |
| CDCA3* | 83461 | Hs00229905_m1 | NM_031299.4 |
| CDCA8* | 55143 | Hs00983655_m1 | NM_018101.2 |
| CDKN3* | 1033 | Hs00193192_m1 | NM_001130851.1; |
| | | | NM_005192.3 |
| CDT1* | 81620 | Hs00368864_m1 | NM_030928.3 |
| CENPA | 1058 | Hs00156455_m1 | NM_001042426.1; |
| | | | NM_001809.3 |
| CENPE* | 1062 | Hs00156507_m1 | NM_001813.2 |
| CENPF* | 1063 | Hs00193201_m1 | NM_016343.3 |
| CENPI* | 2491 | Hs00198791_m1 | NM_006733.2 |
| CENPM* | 79019 | Hs00608780_m1 | NM_024053.3 |
| CENPN* | 55839 | Hs00218401_m1 | NM_018455.4; |
| | | | NM_001100624.1; |
| | | | NM_001100625.1 |
| CEP55* | 55165 | Hs00216688_m1 | NM_018131.4; |
| | | | NM_001127182.1 |
| CHEK1* | 1111 | Hs00967506_ml | NM_001114121.1; |
| | | | NM_001114122.1; |
| | | | NM_001274.4 |
| CKAP2* | 26586 | Hs00217068_m1 | NM_018204.3; |
| | | | NM_001098525.1 |
| CKS1B* | 1163 | Hs01029137_g1 | NM_001826.2 |
| CKS2* | 1164 | Hs01048812_g1 | NM_001827.1 |
| CTPS* | 1503 | Hs01041851_m1 | NM_001905.2 |
| CTSL2* | 1515 | Hs00952036_m1 | NM_001333.2 |
| DBF4* | 10926 | Hs00272696_m1 | NM_006716.3 |
| DDX39* | 10212 | Hs00271794_m1 | NM_005804.2 |
| DLGAP5/DLG7* | 9787 | Hs00207323_m1 | NM_014750.3 |
| DONSON* | 29980 | Hs00375083_m1 | NM_017613.2 |
| DSN1* | 79980 | Hs00227760_m1 | NM_024918.2 |
| DTL* | 51514 | Hs00978565_m1 | NM_016448.2 |
| E2F8* | 79733 | Hs00226635_m1 | NM_024680.2 |
| ECT2* | 1894 | Hs00216455_m1 | NM_018098.4 |
| ESPL1* | 9700 | Hs00202246_m1 | NM_012291.4 |
| EXO1* | 9156 | Hs00243513_m1 | NM_130398.2; |
| | | | NM_003686.3; |
| | | | NM_006027.3 |
| EZH2* | 2146 | Hs00544830_m1 | NM_152998.1; |
| | | | NM_004456.3 |
| FANCI* | 55215 | Hs00289551_m1 | NM_018193.2; |
| | | | NM_001113378.1 |
| FBXO5* | 26271 | Hs03070834_m1 | NM_001142522.1; |
| | | | NM_012177.3 |
| FOXM1* | 2305 | Hs01073586_m1 | NM_202003.1; |
| | | | NM_202002.1; |
| | | | NM_021953.2 |
| GINS1* | 9837 | Hs00221421_m1 | NM_021067.3 |
| GMPS* | 8833 | Hs00269500_m1 | NM_003875.2 |
| GPSM2* | 29899 | Hs00203271_m1 | NM_013296.4 |
| GTSE1* | 51512 | Hs00212681_m1 | NM_016426.5 |
| H2AFX* | 3014 | Hs00266783_s1 | NM_002105.2 |
| HMMR* | 3161 | Hs00234864_m1 | NM_001142556.1; |
| | | | NM_001142557.1; |
| | | | NM_012484.2; |
| | | | NM_012485.2 |
| HN1* | 51155 | Hs00602957_m1 | NM_001002033.1; |
| | | | NM_001002032.1; |
| | | | NM_016185.2 |
| KIAA0101* | 9768 | Hs00207134_m1 | NM_014736.4 |
| KIF11* | 3832 | Hs00189698_m1 | NM_004523.3 |
| KIF15* | 56992 | Hs00173349_m1 | NM_020242.2 |
| KIF18A* | 81930 | Hs01015428_m1 | NM_031217.3 |
| KIF20A* | 10112 | Hs00993573_m1 | NM_005733.2 |
| KIF20B/MPHOSPH1* | 9585 | Hs01027505_m1 | NM_016195.2 |
| KIF23* | 9493 | Hs00370852_m1 | NM_138555.1; |
| | | | NM_004856.4 |
| KIF2C* | 11004 | Hs00199232_m1 | NM_006845.3 |
| KIF4A* | 24137 | Hs01020169_m1 | NM_012310.3 |
| KIFC1* | 3833 | Hs00954801_m1 | NM_002263.3 |
| KPNA2 | 3838 | Hs00818252_g1 | NM_002266.2 |
| LMNB2* | 84823 | Hs00383326_m1 | NM_032737.2 |
| MAD2L1 | 4085 | Hs01554513_g1 | NM_002358.3 |
| MCAM* | 4162 | Hs00174838_m1 | NM_006500.2 |
| MCM10* | 55388 | Hs00960349_m1 | NM_018518.3; |
| | | | NM_182751.1 |
| MCM2* | 4171 | Hs00170472_m1 | NM_004526.2 |
| MCM4* | 4173 | Hs00381539_m1 | NM_005914.2; |
| | | | NM_182746.1 |
| MCM6* | 4175 | Hs00195504_m1 | NM_005915.4 |
| MCM7* | 4176 | Hs01097212_m1 | NM_005916.3; |
| | | | NM_182776.1 |
| MELK | 9833 | Hs00207681_m1 | NM_014791.2 |
| MKI67* | 4288 | Hs00606991_m1 | NM_002417.3 |
| MYBL2* | 4605 | Hs00231158_m1 | NM_002466.2 |
| NCAPD2* | 9918 | Hs00274505_m1 | NM_014865.3 |
| NCAPG* | 64151 | Hs00254617_m1 | NM_022346.3 |
| NCAPG2* | 54892 | Hs00375141_m1 | NM_017760.5 |
| NCAPH* | 23397 | Hs01010752_m1 | NM_015341.3 |
| NDC80* | 10403 | Hs00196101_m1 | NM_006101.2 |
| NEK2* | 4751 | Hs00601227_mH | NM_002497.2 |
| NUSAP1* | 51203 | Hs01006195_m1 | NM_018454.6; |
| | | | NM_001129897.1; |
| | | | NM_016359.3 |
| OIP5* | 11339 | Hs00299079_m1 | NM_007280.1 |
| ORC6L* | 23594 | Hs00204876_m1 | NM_014321.2 |
| PAICS* | 10606 | Hs00272390_m1 | NM_001079524.1; |
| | | | NM_001079525.1; |
| | | | NM_006452.3 |
| PBK* | 55872 | Hs00218544_m1 | NM_018492.2 |
| PCNA* | 5111 | Hs00427214_g1 | NM_182649.1; |
| | | | NM_002592.2 |
| PDSS1* | 23590 | Hs00372008_m1 | NM_014317.3 |
| PLK1* | 5347 | Hs00153444_m1 | NM_005030.3 |
| PLK4* | 10733 | Hs00179514_m1 | NM_014264.3 |
| POLE2* | 5427 | Hs00160277_m1 | NM_002692.2 |
| PRC1* | 9055 | Hs00187740_m1 | NM_199413.1; |
| | | | NM_199414.1; |
| | | | NM_003981.2 |
| PSMA7* | 5688 | Hs00895424_m1 | NM_002792.2 |
| PSRC1* | 84722 | Hs00364137_m1 | NM_032636.6; |
| | | | NM_001005290.2; |
| | | | NM_001032290.1; |
| | | | NM_001032291.1 |
| PTTG1* | 9232 | Hs00851754_u1 | NM_004219.2 |
| RACGAP1* | 29127 | Hs00374747_m1 | NM_013277.3 |
| RAD51* | 5888 | Hs00153418_m1 | NM_133487.2; |
| | | | NM_002875.3 |
| RAD51AP1* | 10635 | Hs01548891_m1 | NM_001130862.1; |
| | | | NM_006479.4 |
| RAD54B* | 25788 | Hs00610716_m1 | NM_012415.2 |
| RAD54L* | 8438 | Hs00269177_m1 | NM_001142548.1; |
| | | | NM_003579.3 |
| RFC2* | 5982 | Hs00945948_m1 | NM_181471.1; |
| | | | NM_002914.3 |
| RFC4* | 5984 | Hs00427469_m1 | NM_181573.2; |
| | | | NM_002916.3 |
| RFC5* | 5985 | Hs00738859_m1 | NM_181578.2; |
| | | | NM_001130112.1; |
| | | | NM_001130113.1; |
| | | | NM_007370.4 |
| RNASEH2A* | 10535 | Hs00197370_m1 | NM_006397.2 |
| RRM2* | 6241 | Hs00357247_g1 | NM_001034.2 |
| SHCBP1* | 79801 | Hs00226915_m1 | NM_024745.4 |
| SMC2* | 10592 | Hs00197593_m1 | NM_001042550.1; |
| | | | NM_001042551.1; |
| | | | NM_006444.2 |
| SPAG5* | 10615 | Hs00197708_m1 | NM_006461.3 |
| SPC25* | 57405 | Hs00221100_m1 | NM_020675.3 |
| STIL* | 6491 | Hs00161700_m1 | NM_001048166.1; |
| | | | NM_003035.2 |
| STMN1* | 3925 | Hs00606370_m1 | NM_005563.3; |
| | | Hs01033129_m1 | NM_203399.1 |
| TACC3* | 10460 | Hs00170751_m1 | NM_006342.1 |
| TIMELESS* | 8914 | Hs01086966_m1 | NM_003920.2 |
| TK1* | 7083 | Hs01062125_m1 | NM_003258.4 |
| TOP2A* | 7153 | Hs00172214_m1 | NM_001067.2 |
| TPX2* | 22974 | Hs00201616_m1 | NM_012112.4 |
| TRIP13* | 9319 | Hs01020073_m1 | NM_004237.2 |
| TTK* | 7272 | Hs00177412_m1 | NM_003318.3 |
| TUBA1C* | 84790 | Hs00733770_m1 | NM_032704.3 |
| TYMS* | 7298 | Hs00426591_m1 | NM_001071.2 |
| UBE2C | 11065 | Hs00964100_g1 | NM_181799.1; |
| | | | NM_181800.1; |
| | | | NM_181801.1; |
| | | | NM_181802.1; |
| | | | NM_181803.1; |
| | | | NM_007019.2 |
| UBE2S | 27338 | Hs00819350_m1 | NM_014501.2 |
| VRK1* | 7443 | Hs00177470_m1 | NM_003384.2 |
| ZWILCH* | 55055 | Hs01555249_m1 | NM_017975.3; |
| | | | NR_003105.1 |
| ZWINT* | 11130 | Hs00199952_m1 | NM_032997.2; |
| | | | NM_001005413.1; |
| | | | NM_007057.3 |

*124-gene subset of CCP genes useful in the invention (“Panel B”). ABI Assay ID means the catalogue ID number for the gene expression assay commercially available from Applied Biosystems Inc. (Foster City, CA) for the particular gene.

Additional CCP gene panels useful in the invention are as follows:

TABLE 3
“Panel C”

| Gene | Entrez | Gene | Entrez | Gene | Entrez |
|---|---|---|---|---|---|
| Symbol | GeneID | Symbol | GeneID | Symbol | GeneID |
| AURKA | 6790 | DTL* | 51514 | PRC1* | 9055 |
| BUB1* | 699 | FOXM1* | 2305 | PTTG1* | 9232 |
| CCNB1* | 891 | HMMR* | 3161 | RRM2* | 6241 |
| CCNB2* | 9133 | KIF23* | 9493 | TIMELESS* | 8914 |
| CDC2* | 983 | KPNA2 | 3838 | TPX2* | 22974 |
| CDC20* | 991 | MAD2L1* | 4085 | TRIP13* | 9319 |
| CDC45L* | 8318 | MELK | 9833 | TTK* | 7272 |
| CDCA8* | 55143 | MYBL2* | 4605 | UBE2C | 11065 |
| CENPA | 1058 | NUSAP1* | 51203 | UBE2S* | 27338 |
| CKS2* | 1164 | PBK* | 55872 | ZWINT* | 11130 |
| DLG7* | 9787 | | | | |

*These genes are useful as a 26-gene subset panel (“Panel D”).

TABLE 4
“Panel E”

| Gene | Entrez | Gene | Entrez | Gene | Entrez |
|---|---|---|---|---|---|
| Symbol | GeneID | Symbol | GeneID | Symbol | GeneID |
| ASF1B* | 55723 | CENPM* | 79019 | ORC6L* | 23594 |
| ASPM* | 259266 | CEP55* | 55165 | PBK* | 55872 |
| BIRC5* | 332 | DLGAP5* | 9787 | PLK1* | 5347 |
| BUB1B* | 701 | DTL* | 51514 | PRC1* | 9055 |
| C18orf24* | 220134 | FOXM1* | 2305 | PTTG1* | 9232 |
| CDC2* | 983 | KIAA0101* | 9768 | RAD51* | 5888 |
| CDC20* | 991 | KIF11* | 3832 | RAD54L* | 8438 |
| CDCA3* | 83461 | KIF20A* | 10112 | RRM2* | 6241 |
| CDCA8* | 55143 | KIF4A | 24137 | TK1* | 7083 |
| CDKN3* | 1033 | MCM10* | 55388 | TOP2A* | 7153 |
| CENPF* | 1063 | NUSAP1* | 51203 | | |

*These genes are useful as a 31-gene subset panel (“Panel F”).

TABLE 5
“Panel G”

| Gene | Entrez | | Entrez | Gene | Entrez |
|---|---|---|---|---|---|
| Symbol | GeneID | Gene Symbol | GeneID | Symbol | GeneID |
| AURKA | 6790 | DLG7/DLGAP5 | 9787 | PBK | 55872 |
| BUB1 | 699 | DTL | 51514 | PRC1 | 9055 |
| CCNB1 | 891 | FOXM1 | 2305 | PTTG1 | 9232 |
| CCNB2 | 9133 | HMMR | 3161 | RRM2 | 6241 |
| CDC2/CDK1 | 983 | KIF23 | 9493 | TPX2 | 22974 |
| CDC20 | 991 | MAD2L1 | 4085 | TRIP13 | 9319 |
| CDC45L | 8318 | MELK | 9833 | TTK | 7272 |
| CDCA8 | 55143 | MYBL2 | 4605 | UBE2C | 11065 |
| CENPA | 1058 | NUSAP1 | 51203 | ZWINT | 11130 |
| CKS2 | 1164 | | | | |

Various embodiments of the invention involve determining the expression of genes (e.g., BRCA1, BRCA2, CCP genes, etc.) in a sample. In the context of an individual test gene, “expression level” means the amount (normalized or absolute) of an analyte associated with that gene in a sample. For example, the level of BRCA1 expression can be the amount of BRCA1 transcript (or cDNA reverse transcribed from such transcript) or protein in a sample.

Those skilled in the art are familiar with various techniques for determining the expression level of a gene or protein in a tissue or cell sample. Gene expression can be determined either at the RNA level (i.e., noncoding RNA (ncRNA), mRNA, miRNA, tRNA, rRNA, snoRNA, siRNA and piRNA) or at the protein level. Expression analysis at the RNA level can be done using, e.g., microarray analysis (e.g., for assaying mRNA or microRNA expression, copy number, etc.), quantitative real-time PCR™ (“qRT-PCR™”, e.g., TaqMan™), etc. Levels of proteins in a tumor sample can be determined by any known techniques in the art, e.g., HPLC, mass spectrometry, or using antibodies specific to selected proteins (e.g., IHC, ELISA, etc.). The activity level of a polypeptide encoded by a gene may be used in much the same way as the expression level of the gene or polypeptide. Often higher activity levels indicate higher expression levels while lower activity levels indicate lower expression levels. Thus, in some embodiments, the activity level of a polypeptide encoded by a gene is determined rather than or in addition to the expression level of the gene. Those skilled in the art are familiar with techniques for measuring the activity of various such proteins, including BRCA1, BRCA2, and those encoded by the genes listed in Tables 1 to 5. The methods of the invention may be practiced independent of the particular technique used.

In some embodiments, the expression of one or more normalizing genes is also obtained for use in normalizing the expression of test genes. As used herein, “normalizing genes” referred to the genes whose expression is used to calibrate or normalize the measured expression of the gene of interest (e.g., test genes). Importantly, the expression of normalizing genes should be independent of cancer outcome/prognosis, and the expression of the normalizing genes is very similar among all the tumor samples. Normalization ensures accurate comparison of expression of a test gene between different samples. For this purpose, housekeeping genes known in the art can be used. Housekeeping genes are well known in the art, with examples including, but are not limited to, GUSB (glucuronidase, beta), HMBS (hydroxymethylbilane synthase), SDHA (succinate dehydrogenase complex, subunit A, flavoprotein), UBC (ubiquitin C) and YWHAZ (tyrosine 3-monooxygenase/tryptophan 5-monooxygenase activation protein, zeta polypeptide). One or more housekeeping genes can be used. Preferably, at least 2, 5, 10 or 15 housekeeping genes are used to provide a combined normalizing gene set. The amount of gene expression of such normalizing genes can be averaged, combined together by straight additions or by a defined algorithm. Some examples of particularly useful housekeeper genes for use in the methods and compositions of the invention include those listed in Table A below.

TABLE A

| Gene | Entrez | Applied Biosystems | |
|---|---|---|---|
| Symbol | GeneID | Assay ID | RefSeq Accession Nos. |
| CLTC* | 1213 | Hs00191535_m1 | NM_004859.3 |
| GUSB | 2990 | Hs99999908_m1 | NM_000181.2 |
| HMBS | 3145 | Hs00609297_m1 | NM_000190.3 |
| MMADHC* | 27249 | Hs00739517_g1 | NM_015702.2 |
| MRFAP1* | 93621 | Hs00738144_g1 | NM_033296.1 |
| PPP2CA* | 5515 | Hs00427259_m1 | NM_002715.2 |
| PSMA1* | 5682 | Hs00267631_m1 | |
| PSMC1* | 5700 | Hs02386942_g1 | NM_002802.2 |
| RPL13A* | 23521 | Hs03043885_g1 | NM_012423.2 |
| RPL37* | 6167 | Hs02340038_g1 | NM_000997.4 |
| RPL38* | 6169 | Hs00605263_g1 | NM_000999.3 |
| RPL4* | 6124 | Hs03044647_g1 | NM_000968.2 |
| RPL8* | 6132 | Hs00361285_g1 | NM_033301.1; |
| | | | NM_000973.3 |
| RPS29* | 6235 | Hs03004310_g1 | NM_001030001.1; |
| | | | NM_001032.3 |
| SDHA | 6389 | Hs00188166_m1 | NM_004168.2 |
| SLC25A3* | 6515 | Hs00358082_m1 | NM_213611.1; |
| | | | NM_002635.2; |
| | | | NM_005888.2 |
| TXNL1* | 9352 | Hs00355488_m1 | NR_024546.1; |
| | | | NM_004786.2 |
| UBA52* | 7311 | Hs03004332_g1 | NM_001033930.1; |
| | | | NM_003333.3 |
| UBC | 7316 | Hs00824723_m1 | NM_021009.4 |
| YWHAZ | 7534 | Hs00237047_m1 | NM_003406.3 |

*Subset of useful housekeeping genes.

In the case of measuring RNA levels for the genes, one convenient and sensitive approach is the real-time quantitative PCR™ (gPCR™) assay, following a reverse transcription reaction. Typically, a cycle threshold (Cₜ) is determined for each test gene and each normalizing gene, i.e., the number of cycles at which the fluoescence from a qPCR reaction above background is detectable.

The overall expression of the one or more normalizing genes can be represented by a “normalizing value” which can be generated by combining the expression of all normalizing genes, either weighted equally (straight addition or averaging) or by different predefined coefficients. In one simple example, the normalizing value C_{tH} can be the cycle threshold (Cₜ) of one single normalizing gene, or an average of the Cₜ values of 2 or more, preferably 10 or more, or 15 or more normalizing genes, in which case, the predefined coefficient is 1/N, where N is the total number of normalizing genes used. Thus, C_{tH}=(C_{tH1}+C_{tH2}+ . . . C_{tHn})/N. As will be apparent to skilled artisans, depending on the normalizing genes used, and the weight desired to be given to each normalizing gene, any coefficients (from 0/N to N/N) can be given to the normalizing genes in weighting the expression of such normalizing genes. That is, C_{tH}=xC_{tH1}+yC_{tH2}+ . . . zC_{tHn}, wherein x+y+ . . . +z=1.

As discussed above, the methods of the invention generally involve determining the level of expression of a panel of CCP genes. With modern high-throughput techniques, it is often possible to determine the expression level of tens, hundreds or thousands of genes. Indeed, it is possible to determine the level of expression of the entire transcriptome (i.e., each transcribed gene in the genome). Once such a global assay has been performed, one may then informatically analyze one or more subsets (i.e., panels) of genes. For example, one may analyze the expression of a panel comprising primarily CCP genes according to the present invention by combining the expression level values of the individual test genes to obtain a test value.

As will be apparent to a skilled artisan, such a test value represents the overall expression level of the panel of test genes (e.g., a panel composed of substantially CCP genes). In one embodiment, to provide a test value in the methods of the invention, the normalized expression for a test gene can be obtained by normalizing the measured Cₜ for the test gene against the C_{tH}, i.e., ΔCₜ₁=(Cₜ₁−C_{tH}). Thus, the test value representing the overall expression of the plurality of test genes can be provided by combining the normalized expression of all test genes, either by straight addition or averaging (i.e., weighted equally) or by a different predefined coefficient. For example, the simplest approach is averaging the normalized expression of all test genes: test value=(ΔCₜ₁+ΔCₜ₂+ . . . +ΔCₜₙ)/n. As will be apparent to skilled artisans, depending on the test genes used, different weight can also be given to different test genes in the present invention.

Thus in methods of the invention described herein comprising determining the expression of a panel of CCP genes, such determining step may comprise: (1) determining the expression of a panel of genes in the sample comprising at least two CCP genes; and (2) providing a test value by (a) weighting the determined expression of each of a plurality of test genes selected from said panel of genes with a predefined coefficient, and (b) combining the weighted expression to provide said test value. This test value represents the level of expression of the panel of genes in the sample. In embodiments involving comparison or analysis of CCP expression, the test value will often be compared to BRCA expression in order to determine whether the two are correlated or anti-correlated. In some embodiments, anti-correlation indicates BRCA deficiency.

In some embodiments the methods of the invention comprise determining the status of a panel (i.e., a plurality) of test genes comprising a plurality of CCP genes (e.g., to provide a test value representing the average expression of the test genes). For example, increased expression in a panel of test genes may refer to the average expression level of all panel genes in a particular patient being higher than the average expression level of these genes in normal patients (or higher than some index value that has been determined to represent the normal average expression level). Alternatively, increased expression in a panel of test genes may refer to increased expression in at least a certain number (e.g., 1, 2, 3, 4, 5, 6, 7, 8, 9, 10, 15, 20, 25, 30 or more) or at least a certain proportion (e.g., 10%, 20%, 30%, 40%, 50%, 60%, 70%, 80%, 90%, 95%, 99%, 100%) of the genes in the panel as compared to the average normal expression level.

In some embodiments the plurality of test genes (which may itself be a sub-panel analyzed informatically) comprises at least 2, 3, 4, 5, 6, 7, 8, 9, 10, 15, 20, 25, 30, 35, 40, 45, 50, 70, 80, 90, 100, 200, or more CCP genes. In some embodiments the plurality of test genes comprises at least 10, 15, 20, or more CCP genes. In some embodiments the plurality of test genes comprises between 5 and 100 CCP genes, between 7 and 40 CCP genes, between 5 and 25 CCP genes, between 10 and 20 CCP genes, or between 10 and 15 CCP genes. In some embodiments CCP genes comprise at least a certain proportion of the plurality of test genes used to provide a test value. Thus in some embodiments the plurality of test genes comprises at least 25%, 30%, 40%, 50%, 60%, 70%, 75%, 80%, 85%, 90%, 95%, 96%, 97%, 98%, 99%, or 100% CCP genes. In some preferred embodiments the plurality of test genes comprises at least 10, 15, 20, 25, 30, 35, 40, 45, 50, 70, 80, 90, 100, 200, or more CCP genes, and such CCP genes constitute at least 50%, 60%, 70%, preferably at least 75%, 80%, 85%, more preferably at least 90%, 95%, 96%, 97%, 98%, or 99% or more of the total number of genes in the plurality of test genes.

In some embodiments the CCP genes are the genes in any one of Table 1 and Panels A through G. In some embodiments the test panel comprises at least 2, 3, 4, 5, 6, 7, 8, 9, 10, 11, 12, 13, 14, 15, 20, 25, 30, or more of the genes in any of Tables 1 to 5 and Panels A to F. In some embodiments the invention provides methods comprising determining (e.g., in a sample) the expression of the genes in any one of Tables 1 to 5 and Panels A to F.

It has been determined that, once the CCP phenomenon reported herein is appreciated, the choice of individual CCGs for a test panel can, in some embodiments, be somewhat arbitrary. In other words, many CCGs have been found to be very good surrogates for each other. Thus any CCG (or panel of CCGs) can be used in the various embodiments of the invention. In other embodiments of the invention, optimized CCGs are used. One way of assessing whether particular CCGs will serve well in the methods and compositions of the invention is by assessing their correlation with the mean expression of CCGs (e.g., all known CCGs, a specific set of CCGs, etc.). Those CCGs that correlate particularly well with the mean are expected to perform well in assays of the invention, e.g., because these will reduce noise in the assay.

126 CCGs and 47 housekeeping genes had their expression compared to the CCG and housekeeping mean in order to determine preferred genes for use in some embodiments of the invention. Rankings of select CCGs according to their correlation with the mean CCG expression as well as their ranking according to predictive value are given in Tables 2, 3, 5, 6, & 7.

Assays of 126 CCGs and 47 HK (housekeeping) genes were run against 96 commercially obtained, anonymous prostate tumor FFPE samples without outcome or other clinical data. The working hypothesis was that the assays would measure with varying degrees of accuracy the same underlying phenomenon (cell cycle proliferation within the tumor for the CCGs, and sample concentration for the HK genes). Assays were ranked by the Pearson's correlation coefficient between the individual gene and the mean of all the candidate genes, that being the best available estimate of biological activity. Rankings for these 126 CCGs according to their correlation to the overall CCG mean are reported in Table 6.

TABLE 6

| Gene | Gene | Correl. |
|---|---|---|
| # | Symbol | w/Mean |
| 1 | TPX2 | 0.931 |
| 2 | CCNB2 | 0.9287 |
| 3 | KIF4A | 0.9163 |
| 4 | KIF2C | 0.9147 |
| 5 | BIRC5 | 0.9077 |
| 6 | BIRC5 | 0.9077 |
| 7 | RACGAP1 | 0.9073 |
| 8 | CDC2 | 0.906 |
| 9 | PRC1 | 0.9053 |
| 10 | DLGAP5/ | 0.9033 |
| | DLG7 | |
| 11 | CEP55 | 0.903 |
| 12 | CCNB1 | 0.9 |
| 13 | TOP2A | 0.8967 |
| 14 | CDC20 | 0.8953 |
| 15 | KIF20A | 0.8927 |
| 16 | BUB1B | 0.8927 |
| 17 | CDKN3 | 0.8887 |
| 18 | NUSAP1 | 0.8873 |
| 19 | CCNA2 | 0.8853 |
| 20 | KIF11 | 0.8723 |
| 21 | CDCA8 | 0.8713 |
| 22 | NCAPG | 0.8707 |
| 23 | ASPM | 0.8703 |
| 24 | FOXM1 | 0.87 |
| 25 | NEK2 | 0.869 |
| 26 | ZWINT | 0.8683 |
| 27 | PTTG1 | 0.8647 |
| 28 | RRM2 | 0.8557 |
| 29 | TTK | 0.8483 |
| 30 | TRIP13 | 0.841 |
| 31 | GINS1 | 0.841 |
| 32 | CENPF | 0.8397 |
| 33 | HMMR | 0.8367 |
| 34 | NCAPH | 0.8353 |
| 35 | NDC80 | 0.8313 |
| 36 | KIF15 | 0.8307 |
| 37 | CENPE | 0.8287 |
| 38 | TYMS | 0.8283 |
| 39 | KIAA0101 | 0.8203 |
| 40 | FANCI | 0.813 |
| 41 | RAD51AP1 | 0.8107 |
| 42 | CKS2 | 0.81 |
| 43 | MCM2 | 0.8063 |
| 44 | PBK | 0.805 |
| 45 | ESPL1 | 0.805 |
| 46 | MKI67 | 0.7993 |
| 47 | SPAG5 | 0.7993 |
| 48 | MCM10 | 0.7963 |
| 49 | MCM6 | 0.7957 |
| 50 | OIP5 | 0.7943 |
| 51 | CDC45L | 0.7937 |
| 52 | KIF23 | 0.7927 |
| 53 | EZH2 | 0.789 |
| 54 | SPC25 | 0.7887 |
| 55 | STIL | 0.7843 |
| 56 | CENPN | 0.783 |
| 57 | GTSE1 | 0.7793 |
| 58 | RAD51 | 0.779 |
| 59 | CDCA3 | 0.7783 |
| 60 | TACC3 | 0.778 |
| 61 | PLK4 | 0.7753 |
| 62 | ASF1B | 0.7733 |
| 63 | DTL | 0.769 |
| 64 | CHEK1 | 0.7673 |
| 65 | NCAPG2 | 0.7667 |
| 66 | PLK1 | 0.7657 |
| 67 | TIMELESS | 0.762 |
| 68 | E2F8 | 0.7587 |
| 69 | EXO1 | 0.758 |
| 70 | ECT2 | 0.744 |
| 71 | STMN1 | 0.737 |
| 72 | STMN1 | 0.737 |
| 73 | RFC4 | 0.737 |
| 74 | CDC6 | 0.7363 |
| 75 | CENPM | 0.7267 |
| 76 | MYBL2 | 0.725 |
| 77 | SHCBP1 | 0.723 |
| 78 | ATAD2 | 0.723 |
| 79 | KIFC1 | 0.7183 |
| 80 | DBF4 | 0.718 |
| 81 | CKS1B | 0.712 |
| 82 | PCNA | 0.7103 |
| 83 | FBXO5 | 0.7053 |
| 84 | C12orf48 | 0.7027 |
| 85 | TK1 | 0.7017 |
| 86 | BLM | 0.701 |
| 87 | KIF18A | 0.6987 |
| 88 | DONSON | 0.688 |
| 89 | MCM4 | 0.686 |
| 90 | RAD54B | 0.679 |
| 91 | RNASEH2A | 0.6733 |
| 92 | TUBA1C | 0.6697 |
| 93 | C18orf24 | 0.6697 |
| 94 | SMC2 | 0.6697 |
| 95 | CENPI | 0.6697 |
| 96 | GMPS | 0.6683 |
| 97 | DDX39 | 0.6673 |
| 98 | POLE2 | 0.6583 |
| 99 | APOBEC3B | 0.6513 |
| 100 | RFC2 | 0.648 |
| 101 | PSMA7 | 0.6473 |
| 102 | MPHOSPH1/ | 0.6457 |
| | kif20b | |
| 103 | CDT1 | 0.645 |
| 104 | H2AFX | 0.6387 |
| 105 | ORC6L | 0.634 |
| 106 | C1orf135 | 0.6333 |
| 107 | PSRC1 | 0.633 |
| 108 | VRK1 | 0.6323 |
| 109 | CKAP2 | 0.6307 |
| 110 | CCDC99 | 0.6303 |
| 111 | CCNE1 | 0.6283 |
| 112 | LMNB2 | 0.625 |
| 113 | GPSM2 | 0.625 |
| 114 | PAICS | 0.6243 |
| 115 | MCAM | 0.6227 |
| 116 | DSN1 | 0.622 |
| 117 | NCAPD2 | 0.6213 |
| 118 | RAD54L | 0.6213 |
| 119 | PDSS1 | 0.6203 |
| 120 | HN1 | 0.62 |
| 121 | C21orf45 | 0.6193 |
| 122 | CTSL2 | 0.619 |
| 123 | CTPS | 0.6183 |
| 124 | MCM7 | 0.618 |
| 125 | ZWILCH | 0.618 |
| 126 | RFC5 | 0.6177 |

After excluding CCGs with low average expression, assays that produced sample failures, CCGs with correlations less than 0.58, and HK genes with correlations less than 0.95, a subset of 56 CCGs (Panel H) and 36 HK candidate genes were left. Correlation coefficients were recalculated on these subsets, with the rankings shown in Tables 7 and 8, respectively.

TABLE 7
(“Panel H”)

| | | Correl. |
|---|---|---|
| Gene | Gene | w/CCG |
| # | Symbol | mean |
| 1 | FOXM1 | 0.908 |
| 2 | CDC20 | 0.907 |
| 3 | CDKN3 | 0.9 |
| 4 | CDC2 | 0.899 |
| 5 | KIF11 | 0.898 |
| 6 | KIAA0101 | 0.89 |
| 7 | NUSAP1 | 0.887 |
| 8 | CENPF | 0.882 |
| 9 | ASPM | 0.879 |
| 10 | BUB1B | 0.879 |
| 11 | RRM2 | 0.876 |
| 12 | DLGAP5 | 0.875 |
| 13 | BIRC5 | 0.864 |
| 14 | KIF20A | 0.86 |
| 15 | PLK1 | 0.86 |
| 16 | TOP2A | 0.851 |
| 17 | TK1 | 0.837 |
| 18 | PBK | 0.831 |
| 19 | ASF1B | 0.827 |
| 20 | C18orf24 | 0.817 |
| 21 | RAD54L | 0.816 |
| 22 | PTTG1 | 0.814 |
| 23 | KIF4A | 0.814 |
| 24 | CDCA3 | 0.811 |
| 25 | MCM10 | 0.802 |
| 26 | PRC1 | 0.79 |
| 27 | DTL | 0.788 |
| 28 | CEP55 | 0.787 |
| 29 | RAD51 | 0.783 |
| 30 | CENPM | 0.781 |
| 31 | CDCA8 | 0.774 |
| 32 | OIP5 | 0.773 |
| 33 | SHCBP1 | 0.762 |
| 34 | ORC6L | 0.736 |
| 35 | CCNB1 | 0.727 |
| 36 | CHEK1 | 0.723 |
| 37 | TACC3 | 0.722 |
| 38 | MCM4 | 0.703 |
| 39 | FANCI | 0.702 |
| 40 | KIF15 | 0.701 |
| 41 | PLK4 | 0.688 |
| 42 | APOBEC3B | 0.67 |
| 43 | NCAPG | 0.667 |
| 44 | TRIP13 | 0.653 |
| 45 | KIF23 | 0.652 |
| 46 | NCAPH | 0.649 |
| 47 | TYMS | 0.648 |
| 48 | GINS1 | 0.639 |
| 49 | STMN1 | 0.63 |
| 50 | ZWINT | 0.621 |
| 51 | BLM | 0.62 |
| 52 | TTK | 0.62 |
| 53 | CDC6 | 0.619 |
| 54 | KIF2C | 0.596 |
| 55 | RAD51AP1 | 0.567 |
| 56 | NCAPG2 | 0.535 |

TABLE 8

| | | Correlation |
|---|---|---|
| Gene | Gene | with HK |
| # | Symbol | Mean |
| 1 | RPL38 | 0.989 |
| 2 | UBA52 | 0.986 |
| 3 | PSMC1 | 0.985 |
| 4 | RPL4 | 0.984 |
| 5 | RPL37 | 0.983 |
| 6 | RPS29 | 0.983 |
| 7 | SLC25A3 | 0.982 |
| 8 | CLTC | 0.981 |
| 9 | TXNL1 | 0.98 |
| 10 | PSMA1 | 0.98 |
| 11 | RPL8 | 0.98 |
| 12 | MMADHC | 0.979 |
| 13 | RPL13A; | 0.979 |
| | LOC728658 | |
| 14 | PPP2CA | 0.978 |
| 15 | MRFAP1 | 0.978 |

The CCGs in Panel F were likewise ranked according to correlation to the CCG mean as shown in Table 9 below.

TABLE 9

| | | Correl. |
|---|---|---|
| Gene | Gene | w/CCG |
| # | Symbol | mean |
| 1 | DLGAP5 | 0.931 |
| 2 | ASPM | 0.931 |
| 3 | KIF11 | 0.926 |
| 4 | BIRC5 | 0.916 |
| 5 | CDCA8 | 0.902 |
| 6 | CDC20 | 0.9 |
| 7 | MCM10 | 0.899 |
| 8 | PRC1 | 0.895 |
| 9 | BUB1B | 0.892 |
| 10 | FOXM1 | 0.889 |
| 11 | NUSAP1 | 0.888 |
| 12 | C18orf24 | 0.885 |
| 13 | PLK1 | 0.879 |
| 14 | CDKN3 | 0.874 |
| 15 | RRM2 | 0.871 |
| 16 | RAD51 | 0.864 |
| 17 | CEP55 | 0.862 |
| 18 | ORC6L | 0.86 |
| 19 | RAD54L | 0.86 |
| 20 | CDC2 | 0.858 |
| 21 | CENPF | 0.855 |
| 22 | TOP2A | 0.852 |
| 23 | KIF20A | 0.851 |
| 24 | KIAA0101 | 0.839 |
| 25 | CDCA3 | 0.835 |
| 26 | ASF1B | 0.797 |
| 27 | CENPM | 0.786 |
| 28 | TK1 | 0.783 |
| 29 | PBK | 0.775 |
| 30 | PTTG1 | 0.751 |
| 31 | DTL | 0.737 |

When choosing specific CCGs for inclusion in any embodiment of the invention, the individual predictive power of each gene may be used to rank them in importance. The inventors have determined that the CCGs in Panel C can be ranked as shown in Table 10 below according to the predictive power of each individual gene. The CCGs in Panel F can be similarly ranked as shown in Table 11 below.

TABLE 10

| Gene | | |
|---|---|---|
| # | Gene | p-value |
| 1 | NUSAP1 | 2.8E−07 |
| 2 | DLG7 | 5.9E−07 |
| 3 | CDC2 | 6.0E−07 |
| 4 | FOXM1 | 1.1E−06 |
| 5 | MYBL2 | 1.1E−06 |
| 6 | CDCA8 | 3.3E−06 |
| 7 | CDC20 | 3.8E−06 |
| 8 | RRM2 | 7.2E−06 |
| 9 | PTTG1 | 1.8E−05 |
| 10 | CCNB2 | 5.2E−05 |
| 11 | HMMR | 5.2E−05 |
| 12 | BUB1 | 8.3E−05 |
| 13 | PBK | 1.2E−04 |
| 14 | TTK | 3.2E−04 |
| 15 | CDC45L | 7.7E−04 |
| 16 | PRC1 | 1.2E−03 |
| 17 | DTL | 1.4E−03 |
| 18 | CCNB1 | 1.5E−03 |
| 19 | TPX2 | 1.9E−03 |
| 20 | ZWINT | 9.3E−03 |
| 21 | KIF23 | 1.1E−02 |
| 22 | TRIP13 | 1.7E−02 |
| 23 | KPNA2 | 2.0E−02 |
| 24 | UBE2C | 2.2E−02 |
| 25 | MELK | 2.5E−02 |
| 26 | CENPA | 2.9E−02 |
| 27 | CKS2 | 5.7E−02 |
| 28 | MAD2L1 | 1.7E−01 |
| 29 | UBE2S | 2.0E−01 |
| 30 | AURKA | 4.8E−01 |
| 31 | TIMELESS | 4.8E−01 |

TABLE 11

| Gene | Gene | |
|---|---|---|
| # | Symbol | p-value |
| 1 | MCM10 | 8.60E−10 |
| 2 | ASPM | 2.30E−09 |
| 3 | DLGAP5 | 1.20E−08 |
| 4 | CENPF | 1.40E−08 |
| 5 | CDC20 | 2.10E−08 |
| 6 | FOXM1 | 3.40E−07 |
| 7 | TOP2A | 4.30E−07 |
| 8 | NUSAP1 | 4.70E−07 |
| 9 | CDKN3 | 5.50E−07 |
| 10 | KIF11 | 6.30E−06 |
| 11 | KIF20A | 6.50E−06 |
| 12 | BUB1B | 1.10E−05 |
| 13 | RAD54L | 1.40E−05 |
| 14 | CEP55 | 2.60E−05 |
| 15 | CDCA8 | 3.10E−05 |
| 16 | TK1 | 3.30E−05 |
| 17 | DTL | 3.60E−05 |
| 18 | PRC1 | 3.90E−05 |
| 19 | PTTG1 | 4.10E−05 |
| 20 | CDC2 | 0.00013 |
| 21 | ORC6L | 0.00017 |
| 22 | PLK1 | 0.0005 |
| 23 | C18orf24 | 0.0011 |
| 24 | BIRC5 | 0.00118 |
| 25 | RRM2 | 0.00255 |
| 26 | CENPM | 0.0027 |
| 27 | RAD51 | 0.0028 |
| 28 | KIAA0101 | 0.00348 |
| 29 | CDCA3 | 0.00863 |
| 30 | PBK | 0.00923 |
| 31 | ASF1B | 0.00936 |

Thus, in some embodiments of each of the various aspects of the invention the plurality of test genes comprises the top 2, 3, 4, 5, 6, 7, 8, 9, 10, 11, 12, 13, 14, 15, 20, 25, 30, 35, 40 or more genes listed in Table 6, 7, 9, 10, or 11. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises at least 1, 2, 3, 4, 5, 6, 7, 8, 9, 10, 15, or of the following genes: ASPM, BIRC5, BUB1B, CCNB2, CDC2, CDC20, CDCA8, CDKN3, CENPF, DLGAP5, FOXM1, KIAA0101, KIF11, KIF2C, KIF4A, MCM10, NUSAP1, PRC1, RACGAP1, and TPX2. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises at least 1, 2, 3, 4, 5, 6, 7, 8, 9, 10, 15, or 20 of the following genes: TPX2, CCNB2, KIF4A, KIF2C, BIRC5, RACGAP1, CDC2, PRC1, DLGAP5/DLG7, CEP55, CCNB1, TOP2A, CDC20, KIF20A, BUB1B, CDKN3, NUSAP1, CCNA2, KIF11, and CDCA8. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, eight, nine, or ten or all of gene numbers 1 & 2, 1 to 3, 1 to 4, 1 to 5, 1 to 6, 1 to 7, 1 to 8, 1 to 9, or 1 to 10 of any of Table 6, 7, 9, 10, or 11. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, eight, or nine or all of gene numbers 2 & 3, 2 to 4, 2 to 5, 2 to 6, 2 to 7, 2 to 8, 2 to 9, or 2 to 10 of any of Table 6, 7, 9, 10, or 11. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, or eight or all of gene numbers 3 & 4, 3 to 5, 3 to 6, 3 to 7, 3 to 8, 3 to 9, or 3 to 10 of any of Table 6, 7, 9, 10, or 11. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, or seven or all of gene numbers 4 & 5, 4 to 6, 4 to 7, 4 to 8, 4 to 9, or 4 to 10 of any of Table 6, 7, 9, 10, or 11. In some embodiments the plurality of test genes comprises at least some number of CCGs (e.g., at least 3, 4, 5, 6, 7, 8, 9, 10, 15, 20, 25, 30, 35, 40, 45, 50 or more CCGs) and this plurality of CCGs comprises any one, two, three, four, five, six, seven, eight, nine, 10, 11, 12, 13, 14, or 15 or all of gene numbers 1 & 2, 1 to 3, 1 to 4, 1 to 5, 1 to 6, 1 to 7, 1 to 8, 1 to 9, 1 to 10, 1 to 11, 1 to 12, 1 to 13, 1 to 14, or 1 to 15 of any of Table 6, 7, 9, 10, or 11.

In CCP signatures the particular CCP genes analyzed is often not as important as the total number of CCP genes. The number of CCP genes analyzed can vary depending on many factors, e.g., technical constraints, cost considerations, the classification being made, the cancer being tested, the desired level of predictive power, etc. Increasing the number of CCP genes analyzed in a panel according to the invention is, as a general matter, advantageous because, e.g., a larger pool of genes to be analyzed means less “noise” caused by outliers and less chance of an error in measurement or analysis throwing off the overall predictive power of the test. However, cost and other considerations will sometimes limit this number and finding the optimal number of CCP genes for a signature is desirable.

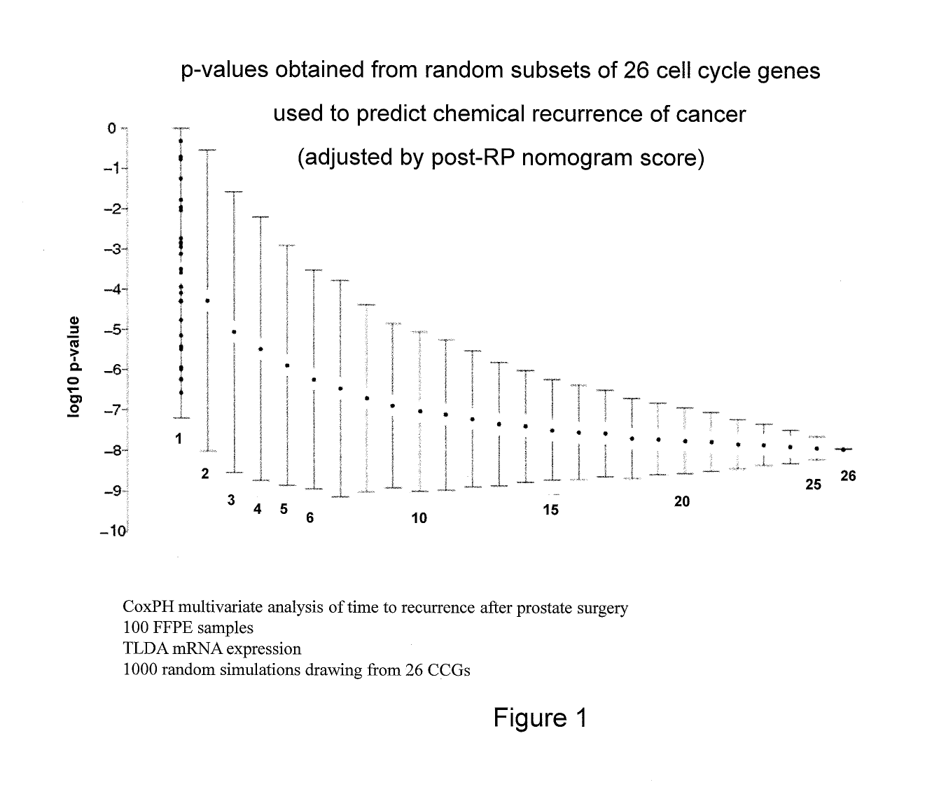

It has been discovered that the predictive power of a CCP signature often ceases to increase significantly beyond a certain number of CCP genes (see FIG. 1; Example 1). More specifically, the optimal number of CCP genes in a signature (n_{O}) can be found wherever the following is true

(Pₙ₊₁−Pₙ)<C_{O},

wherein P is the predictive power (i.e., Pₙ is the predictive power of a signature with n genes and Pₙ₊₁ is the predictive power of a signature with n genes plus one) and C_{O} is some optimization constant. Predictive power can be defined in many ways known to those skilled in the art including, but not limited to, the signature's p-value. C_{O} can be chosen by the artisan based on his or her specific constraints. For example, if cost is not a critical factor and extremely high levels of sensitivity and specificity are desired, C_{O} can be set very low such that only trivial increases in predictive power are disregarded. On the other hand, if cost is decisive and moderate levels of sensitivity and specificity are acceptable, C_{O} can be set higher such that only significant increases in predictive power warrant increasing the number of genes in the signature.

Alternatively, a graph of predictive power as a function of gene number may be plotted (as in FIG. 1) and the second derivative of this plot taken. The point at which the second derivative decreases to some predetermined value (C_{O}′) may be the optimal number of genes in the signature.

Example 1 and FIG. 1 illustrate the empirical determination of optimal numbers of CCP genes in CCP panels of the invention. Randomly selected subsets of the 31 CCP genes listed in Table 3 were tested as distinct CCP signatures and predictive power (i.e., p-value) for predicting prostate cancer recurrence was determined for each. As FIG. 1 shows, p-values ceased to improve significantly beyond about 10 to 15 CCP genes, thus indicating that a preferred number of CCP genes in a diagnostic or prognostic panel is from about 10 to about 15. Thus some embodiments of the invention provide methods comprising determining the expression of a panel of genes, wherein the panel comprises between about 10 and about 15 CCP genes. In some embodiments the panel comprises between about 10 and about 15 CCP genes and the CCP genes constitute at least 25%, 30%, 40%, 50%, 60%, 70%, 75%, 80%, 85%, 90%, 95%, 96%, 97%, 98%, 99%, or 100% of the panel. Any other combination of CCP genes (including any of those listed in Table 1 or Panels A through G) can be used to practice the invention.

Determining expression levels can be, to varying degrees, quantitative, qualitative, or both. For example, when determining the BRCA1 mRNA transcript levels in a sample, the absolute number of transcripts can be determined. Alternatively, the absolute number of transcripts may be normalized against some standard as discussed above to yield a relative rather than absolute expression level. When determining protein expression levels, more qualitative analysis is common. For example, tissue samples may be stained with an antibody against BRCA1 protein and the level of staining in tumor cells can be assigned certain semi-quantitative numbers (e.g., −1, 0, +1). Assigning particular expression levels in this way will often be based on an internal control (e.g., surrounding non-tumor cells) or an external control (e.g., unrelated BRCA-intact cells).

Those skilled in the art are familiar with various ways of determining the expression of a panel (plurality) of genes (e.g., CCP genes). One may determine the expression of a panel of genes by determining the average (e.g., mean, median, weighted average, etc.) expression level, normalized or absolute, of panel genes in a sample obtained from a particular patient (either throughout the sample or in a subset of cells from the sample or in a single cell). Increased expression in this context will mean the average expression is higher than the average expression level of these genes in normal patients (or higher than some index value, e.g., a value that has been determined to represent the average expression level in a reference population (e.g., patients with cancer or patients with the same cancer)). Alternatively, one may determine the expression of a panel of genes by determining the average expression level (normalized or absolute) of at least a certain number (e.g., 1, 2, 3, 4, 5, 6, 7, 8, 9, 10, 15, 20, 25, 30 or more) or at least a certain proportion (e.g., 10%, 20%, 30%, 40%, 50%, 60%, 70%, 80%, 90%, 95%, 99%, 100%) of the genes in the panel. Alternatively, one may determine the expression of a panel of genes by determining the absolute copy number of the mRNA (or protein) of all the genes in the panel and either total or average these across the genes.

In preferred embodiments, the test value representing the expression level of a test gene (e.g., BRCA1) or a plurality of test genes (e.g., a panel of CCP genes) is compared to one or more reference values (or index values) to determine if expression of the test gene(s) is high, low, average, etc. Once BRCA and CCP expression have thus been determined as high, low, etc., one can, according to the methods of the present invention, determine whether BRCA and CCP expression are correlated or anti-correlated.

Those skilled in the art are familiar with various ways of deriving and using index values. For example, the index value may represent the gene expression levels found in a normal sample obtained from the patient of interest, in which case an expression level (e.g., test value) in the test sample significantly above this index value would indicate high expression in the sample.

Alternatively, the index value may represent the average expression level for a set of individuals from a diverse population or a subset of the population. For example, one may determine the average expression level of a gene or gene panel in a random sampling of patients. This average expression level may be termed the “threshold index value.” In some embodiments of the invention the methods comprise determining whether the expression of one or more test genes is “increased” or “high.” In the context of the invention, “increased” or “high” expression of a test gene means the patient's expression level is either elevated over a normal index value or a threshold index (e.g., by at least some threshold amount (e.g., a standard deviation)) or within the range of expression that has been determined in patients to be high (e.g., top quartile of reference patients).

Alternative index values may be derived by dividing patients into groups based on expression level. For example, one may determine the level of expression of the test gene(s) for a set of patients and group the patients into terciles, quartiles, quintiles, etc. A threshold may be set at the boundary of each group, with test patients being placed into a group (e.g., quartile) depending on which threshold(s) their determined expression exceeds.

Alternatively index values may be determined thusly: In order to assign patients to risk groups (e.g., high likelihood of having cancer, high likelihood of recurrence/progression), a threshold value will be set for the cell cycle mean. The optimal threshold value is selected based on the receiver operating characteristic (ROC) curve, which plots sensitivity vs (1−specificity). For each increment of the cell cycle mean, the sensitivity and specificity of the test is calculated using that value as a threshold. The actual threshold will be the value that optimizes these metrics according to the artisan's requirements (e.g., what degree of sensitivity or specificity is desired, etc.).

As mentioned above, anti-correlation between BRCA and CCP expression indicates BRCA deficiency. Thus in one aspect the invention provides a method for determining whether a sample is BRCA deficient comprising measuring the expression of BRCA1 and/or BRCA2 (BRCA expression) in said sample, measuring the expression of a panel of CCP genes in the sample, and determining whether BRCA expression is correlated to CCP expression. In this context, BRCA and CCP expression are “correlated” in a sample if BRCA and CCP expression are both high, low, or intermediate in the sample. Conversely, BRCA and CCP expression are “anti-correlated” in a sample if one is low while the other is high or if one is either high or low and the other is intermediate in the sample. In a preferred embodiment BRCA and CCP expression are anti-correlated if BRCA (especially BRCA1) expression is low and CCP expression (especially expression of one of the panels in Tables 1 to 5 (e.g., Panels A to F)) is high.

In some embodiments the sample is from a patient having (or suspected of having) ovarian cancer, breast cancer, lung cancer, colon cancer, or prostate cancer, or any combination of these. In some embodiments, the sample is a tumor tissue sample, a blood or blood derivative (e.g., serum, plasma) sample, a urine sample, or any other sample derived from the body of a patient. In some embodiments the sample used to determine expression levels is some derivative of these bodily samples (e.g., an isolate of the RNA, DNA, protein, etc. from a bodily sample).

In some embodiments, the invention provides a method for determining whether a sample is BRCA deficient comprising measuring the expression of BRCA1 and/or BRCA2 (BRCA expression) in said sample, measuring the expression of a panel of CCP genes in the sample, and determining whether BRCA expression is correlated to CCP expression, wherein anti-correlation between BRCA and CCP expression indicates the sample is BRCA deficient.

In some embodiments anti-correlation between BRCA and CCP expression indicates the sample has BRCA hypermethylation. Some embodiments further comprise determining the methylation status and level of a gene or panel of genes (preferably the BRCA1 and/or BRCA2 gene) in the sample. As used herein, “methylation status” is used to indicate the presence or absence or the level or extent of methyl group modification in the polynucleotide of at least one gene. As used herein, “methylation level” is used to indicate the quantitative measurement of methylated DNA for a given gene, defined as the percentage of total DNA copies of that gene that are determined to be methylated, based on quantitative methylation-specific PCR.

Any assay that can be employed to determine the methylation status of the gene or gene panel should suffice for the purposes of the present invention. In general, assays are designed to assess the methylation status of individual genes, or portions thereof. Examples of types of assays used to assess the methylation pattern include, but are not limited to, Southern blotting, single nucleotide primer extension, methylation-specific polymerase chain reaction (MSPCR), restriction landmark genomic scanning for methylation (RLGS-M) and CpG island microarray, single nucleotide primer extension (SNuPE), and combined bisulfite restriction analysis (COBRA). The COBRA technique is disclosed in Xiong & Laird, NUCLEIC ACIDS RES. (1997) 25:2532-2534, which is incorporated by reference. In addition, methylation arrays may also be employed to determine the methylation status of a gene or panel of genes. Methylation arrays are disclosed in Beier et al., ADV. BIOCHEM. ENG. BIOTECHNOL. (2007) 104:1-11, which is incorporated by reference. For example, a method for determining the methylation state of nucleic acids is described in U.S. Pat. No. 6,017,704 which is incorporated by reference. Determining the methylation state of the nucleic acid includes amplifying the nucleic acid by means of oligonucleotide primers that distinguishes between methylated and unmethylated nucleic acids.

In some embodiments the panel of CCP genes comprises at least two (or five, or six, or ten, or 15, or more) CCP genes from any of Tables 1 to 5. In some embodiments the panel of CCP genes comprises at least two (or five, or six, or ten, or 15, or more) CCP genes from any of Tables 1 to 5. In some embodiments the panel of CCP genes comprises the genes listed in Table 4. In some embodiments the panel of CCP genes comprises the genes in Panel F. In some embodiments the panel of CCP genes comprises the genes listed in Table 5.

BRCA deficiency has been found to be correlated with, inter alia, progression-free survival (Example 2). Specifically, BRCA deficient patients show a significantly longer progression-free survival than non-BRCA-deficient patients. Thus in one aspect the invention provides a method of classifying a cancer comprising measuring the expression of BRCA1 and/or BRCA2 (BRCA expression) in said sample and measuring the expression of two or more CCP genes in the sample. Some embodiments further comprise determining whether BRCA expression is correlated to CCP expression. In some embodiments, anti-correlation between BRCA and CCP expression indicates any one of the following: greater likelihood of survival (e.g., progression-free survival, overall survival, etc.), greater likelihood of response to DNA damaging agents (e.g., platinum chemotherapy drugs, etc.), greater likelihood of response to drugs targeting the poly (ADP-ribose) polymerase (PARP) pathway, etc.

As used herein, a patient has an “increased likelihood” of some clinical feature or outcome (e.g., recurrence, progression, response to a particular therapeutic regimen, etc.) if the probability of the patient having the feature or outcome exceeds some reference probability or value. The reference probability may be the probability of the feature or outcome across the general relevant patient population. For example, if the probability of recurrence in the general breast cancer population is X % and a particular patient has been determined by the methods of the present invention to have a probability of recurrence of Y %, and if Y>X, then the patient has an “increased likelihood” of recurrence. Alternatively, as discussed above, a threshold or reference value may be determined and a particular patient's probability of recurrence may be compared to that threshold or reference.

Those skilled in the art are familiar with various techniques for determining gene expression and any technique that determines gene expression can be used in the methods of the invention. In some embodiments gene expression is determined using any of the following techniques: quantitative PCR™ (e.g., TaqMan™), microarray hybridization analysis, quantitative sequencing, etc.

The results of any analyses according to the invention will often be communicated to physicians, genetic counselors and/or patients (or other interested parties such as researchers) in a transmittable form that can be communicated or transmitted to any of the above parties. Such a form can vary and can be tangible or intangible. The results can be embodied in descriptive statements, diagrams, photographs, charts, images or any other visual forms. For example, graphs showing expression or activity level or sequence variation information for various genes can be used in explaining the results. Diagrams showing such information for additional target gene(s) are also useful in indicating some testing results. The statements and visual forms can be recorded on a tangible medium such as papers, computer readable media such as floppy disks, compact disks, etc., or on an intangible medium, e.g., an electronic medium in the form of email or website on internet or intranet. In addition, results can also be recorded in a sound form and transmitted through any suitable medium, e.g., analog or digital cable lines, fiber optic cables, etc., via telephone, facsimile, wireless mobile phone, internet phone and the like.

Thus, the information and data on a test result can be produced anywhere in the world and transmitted to a different location. As an illustrative example, when an expression level, activity level, or sequencing (or genotyping) assay is conducted outside the United States, the information and data on a test result may be generated, cast in a transmittable form as described above, and then imported into the United States. Accordingly, the present invention also encompasses a method for producing a transmittable form of information on at least one of (a) expression level or (b) activity level for at least one patient sample. The method comprises the steps of (1) determining at least one of (a) or (b) above according to methods of the present invention; and (2) embodying the result of the determining step in a transmittable form. The transmittable form is the product of such a method.

Techniques for analyzing such expression, activity, and/or sequence data (indeed any data obtained according to the invention) will often be implemented using hardware, software or a combination thereof in one or more computer systems or other processing systems capable of effectuating such analysis.

Thus one aspect of the present invention provides systems related to the above methods of the invention. In one embodiment the invention provides a system for determining gene expression in a tumor sample, comprising: (1) a sample analyzer for determining the expression levels of BRCA1 and/or BRCA2 and a panel of genes comprising at least two CCP genes in a sample, wherein the sample analyzer contains the sample, mRNA from the sample and expressed from the panel of genes, or cDNA synthesized from said mRNA; (2) a first computer program means for (a) receiving gene expression data on BRCA1 and/or BRCA2, (b) receiving gene expression data on at least two test genes selected from the panel of genes, (b) weighting the determined expression of each of the test genes with a predefined coefficient, and (c) combining the weighted expression to provide a CCP test value representing the expression level of the panel of genes.

As with the methods of the invention, the systems of the invention may be used to determine whether BRCA and/or CCP expression in a sample are high, low, etc. Thus in some embodiments the above system further comprises a computer program means of comparing the expression of BRCA1 and/or BRCA2 to a reference value, wherein expression of BRCA1 and/or BRCA2 above this reference value indicates said BRCA1 and/or BRCA2 expression is high. In some embodiments the above system further comprises a computer program means of comparing the CCP test value to a reference value, wherein a CCP test value above this reference value indicates CCP expression is high.

As with the methods of the invention, the systems of the invention may be used to determine whether BRCA and CCP expression are correlated in a sample. Thus in some embodiments the above system further comprises a computer program means of comparing the expression of BRCA1 and/or BRCA2 to the CCP test value, wherein high expression of BRCA1 and/or BRCA2 coupled with a high CCP test value indicates BRCA and CCP expression are correlated, wherein low expression of BRCA1 and/or BRCA2 coupled with a low CCP test value indicates BRCA and CCP expression are correlated, wherein high expression of BRCA1 and/or BRCA2 coupled with a low CCP test value indicates BRCA and CCP expression are anti-correlated, and wherein low expression of BRCA1 and/or BRCA2 coupled with a high CCP test value indicates BRCA and CCP expression are anti-correlated.

As with the methods of the invention, the systems of the invention may be used to determine whether the sample is BRCA deficient. Thus in some embodiments the above system further comprises a computer program means of receiving data on the correlation between BRCA expression and CCP expression in a patient sample and concluding that the sample is BRCA deficient if BRCA expression and CCP expression are anti-correlated in the sample.

In some embodiments the system comprises a sample analyzer for determining the methylation status of BRCA1 and/or BRCA2. In some embodiments this sample analyzer is the same as the sample analyzer for determining gene expression.

In the systems of the invention, as with the methods of the invention described above, the test genes may comprise at least 2, 3, 4, 5, 6, 7, 8, 9, 10, 15, 20, 25, 30, 35, 40, 45, 50, 70, 80, 90, 100, 200, or more CCP genes. In some embodiments the test genes comprise at least 10, 15, 20, or more CCP genes. In some embodiments the test gene comprises between 5 and 100 CCP genes, between 7 and 40 CCP genes, between 5 and 25 CCP genes, between 10 and 20 CCP genes, or between 10 and 15 CCP genes. In some embodiments CCP genes comprise at least a certain proportion of the test genes used to provide a test value. Thus in some embodiments the test genes comprise at least 25%, 30%, 40%, 50%, 60%, 70%, 75%, 80%, 85%, 90%, 95%, 96%, 97%, 98%, 99%, or 100% CCP genes. In some preferred embodiments the test genes comprise at least 10, 15, 20, 25, 30, 35, 40, 45, 50, 70, 80, 90, 100, 200, or more CCP genes, and such CCP genes constitute at least 50%, 60%, 70%, preferably at least 75%, 80%, 85%, more preferably at least 90%, 95%, 96%, 97%, 98%, or 99% or more of the total number of test genes.

In some embodiments, the system further comprises a display module displaying the comparison between the test value and the one or more reference values, or displaying a result of the comparing step.

In a preferred embodiment, the amount of RNA transcribed from the panel of genes including test genes is measured in the sample. In addition, the amount of RNA of one or more housekeeping genes in the sample is also measured, and used to normalize or calibrate the expression of the test genes, as described above.

The sample analyzer can be any instrument useful in determining gene expression, including, e.g., a sequencing machine, a real-time PCR machine, a microarray instrument, etc. In embodiments comprising a sample analyzer for determining methylation status, such a sample analyzer can be any instrument useful in determining methylation status.

The computer-based analysis function can be implemented in any suitable language and/or browsers. For example, it may be implemented with C language and preferably using object-oriented high-level programming languages such as Visual Basic, SmallTalk, C++, and the like. The application can be written to suit environments such as the Microsoft Windows™ environment including Windows™ 98, Windows™ 2000, Windows™ NT, and the like. In addition, the application can also be written for the MacIntosh™, SUN™, UNIX or LINUX environment. In addition, the functional steps can also be implemented using a universal or platform-independent programming language. Examples of such multi-platform programming languages include, but are not limited to, hypertext markup language (HTML), JAVA™, JavaScript™, Flash programming language, common gateway interface/structured query language (CGI/SQL), practical extraction report language (PERL), AppleScript™ and other system script languages, programming language/structured query language (PL/SQL), and the like. Java™- or JavaScript™-enabled browsers such as HotJava™, Microsoft™ Explorer™, or Netscape™ can be used. When active content web pages are used, they may include Java™ applets or ActiveX™ controls or other active content technologies.

The analysis function can also be embodied in computer program products and used in the systems described above or other computer- or internet-based systems. Accordingly, another aspect of the present invention relates to a computer program product comprising a computer-usable medium having computer-readable program codes or instructions embodied thereon for enabling a processor to carry out gene expression analysis. These computer program instructions may be loaded onto a computer or other programmable apparatus to produce a machine, such that the instructions which execute on the computer or other programmable apparatus create means for implementing the functions or steps described above. These computer program instructions may also be stored in a computer-readable memory or medium that can direct a computer or other programmable apparatus to function in a particular manner, such that the instructions stored in the computer-readable memory or medium produce an article of manufacture including instruction means which implement the analysis. The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions or steps described above.

Some embodiments of the present invention provide a system for determining whether a patient sample is BRCA deficient. Generally speaking, the system comprises (1) computer program means for receiving, storing, and/or retrieving data on the correlation between BRCA and CCP expression in a patient sample; (2) computer program means for querying this patient data; (3) computer program means for concluding whether there is or is not a correlation; and optionally (4) computer program means for outputting/displaying this conclusion. In some embodiments this means for outputting the conclusion may comprise a computer program means for informing a health care professional of the conclusion. In some embodiments the system further comprises a computer program means for receiving, storing, and/or retrieving data on BRCA and CCP expression in a patient sample and a computer program means for determining if BRCA and CCP expression are correlated in such sample.

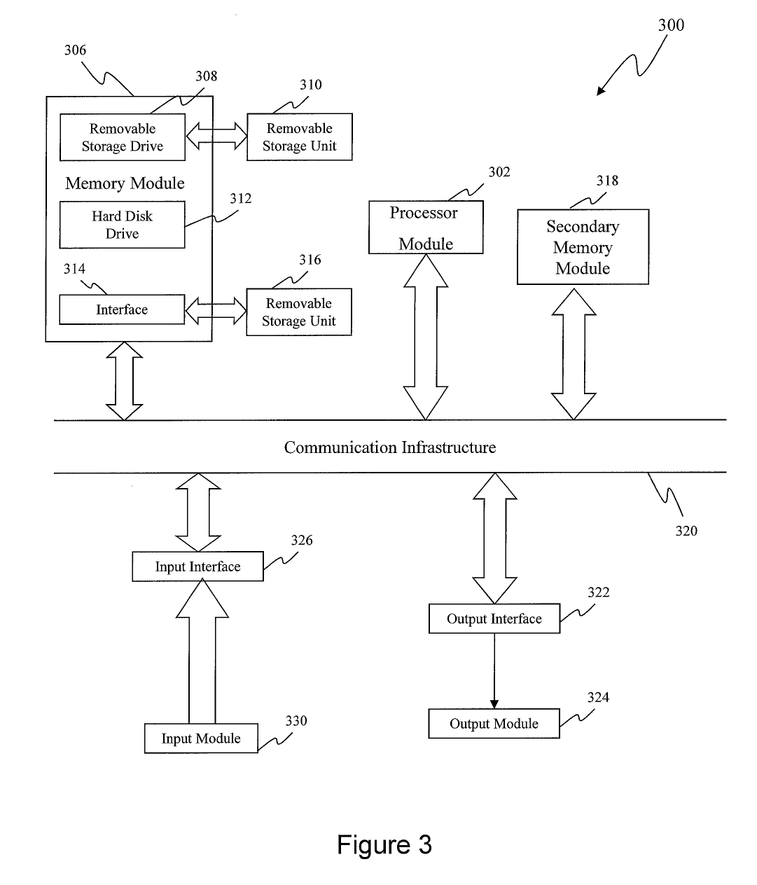

One example of such a computer system is the computer system [300] illustrated in FIG. 3. Computer system [300] may include at least one input module [330] for entering patient data into the computer system [300]. The computer system [300] may include at least one output module [324] for indicating whether a patient has an increased or decreased likelihood of response and/or indicating suggested treatments determined by the computer system [300]. Computer system [300] may include at least one memory module [306] in communication with the at least one input module [330] and the at least one output module [324].

The at least one memory module [306] may include, e.g., a removable storage drive [308], which can be in various forms, including but not limited to, a magnetic tape drive, a floppy disk drive, a VCD drive, a DVD drive, an optical disk drive, etc. The removable storage drive [308] may be compatible with a removable storage unit [310] such that it can read from and/or write to the removable storage unit [310]. Removable storage unit [310] may include a computer usable storage medium having stored therein computer-readable program codes or instructions and/or computer readable data. For example, removable storage unit [310] may store patient data. Example of removable storage unit [310] are well known in the art, including, but not limited to, floppy disks, magnetic tapes, optical disks, and the like. The at least one memory module [306] may also include a hard disk drive [312], which can be used to store computer readable program codes or instructions, and/or computer readable data.

In addition, as shown in FIG. 3, the at least one memory module [306] may further include an interface [314] and a removable storage unit [316] that is compatible with interface [314] such that software, computer readable codes or instructions can be transferred from the removable storage unit [316] into computer system [300]. Examples of interface [314] and removable storage unit [316] pairs include, e.g., removable memory chips (e.g., EPROMs or PROMs) and sockets associated therewith, program cartridges and cartridge interface, and the like. Computer system [300] may also include a secondary memory module [318], such as random access memory (RAM).

Computer system [300] may include at least one processor module [302]. It should be understood that the at least one processor module [302] may consist of any number of devices. The at least one processor module [302] may include a data processing device, such as a microprocessor or microcontroller or a central processing unit. The at least one processor module [302] may include another logic device such as a DMA (Direct Memory Access) processor, an integrated communication processor device, a custom VLSI (Very Large Scale Integration) device or an ASIC (Application Specific Integrated Circuit) device. In addition, the at least one processor module [302] may include any other type of analog or digital circuitry that is designed to perform the processing functions described herein.

As shown in FIG. 3, in computer system [300], the at least one memory module [306], the at least one processor module [302], and secondary memory module [318] are all operably linked together through communication infrastructure [320], which may be a communications bus, system board, cross-bar, etc.). Through the communication infrastructure [320], computer program codes or instructions or computer readable data can be transferred and exchanged. Input interface [326] may operably connect the at least one input module [326] to the communication infrastructure [320]. Likewise, output interface [322] may operably connect the at least one output module [324] to the communication infrastructure [320].

The at least one input module [330] may include, for example, a keyboard, mouse, touch screen, scanner, and other input devices known in the art. The at least one output module [324] may include, for example, a display screen, such as a computer monitor, TV monitor, or the touch screen of the at least one input module [330]; a printer; and audio speakers. Computer system [300] may also include, modems, communication ports, network cards such as Ethernet cards, and newly developed devices for accessing intranets or the internet.

The at least one memory module [306] may be configured for storing patient data entered via the at least one input module [330] and processed via the at least one processor module [302]. Patient data relevant to the present invention may include expression level, activity level, copy number and/or sequence information for a CCP and optionally PTEN. Patient data relevant to the present invention may also include clinical parameters relevant to the patient's disease. Any other patient data a physician might find useful in making treatment decisions/recommendations may also be entered into the system, including but not limited to age, gender, and race/ethnicity and lifestyle data such as diet information. Other possible types of patient data include symptoms currently or previously experienced, patient's history of illnesses, medications, and medical procedures.

The at least one memory module [306] may include a computer-implemented method stored therein. The at least one processor module [302] may be used to execute software or computer-readable instruction codes of the computer-implemented method. The computer-implemented method may be configured to, based upon the patient data, indicate whether the patient has an increased likelihood of recurrence, progression or response to any particular treatment, generate a list of possible treatments, etc.

In certain embodiments, the computer-implemented method may be configured to identify a patient as having or not having cancer or as having or not having an increased likelihood of recurrence or progression. For example, the computer-implemented method may be configured to inform a physician that a particular patient has cancer, has a quantified probability of having cancer, has an increased likelihood of recurrence, etc. Alternatively or additionally, the computer-implemented method may be configured to actually suggest a particular course of treatment based on the answers to/results for various queries.

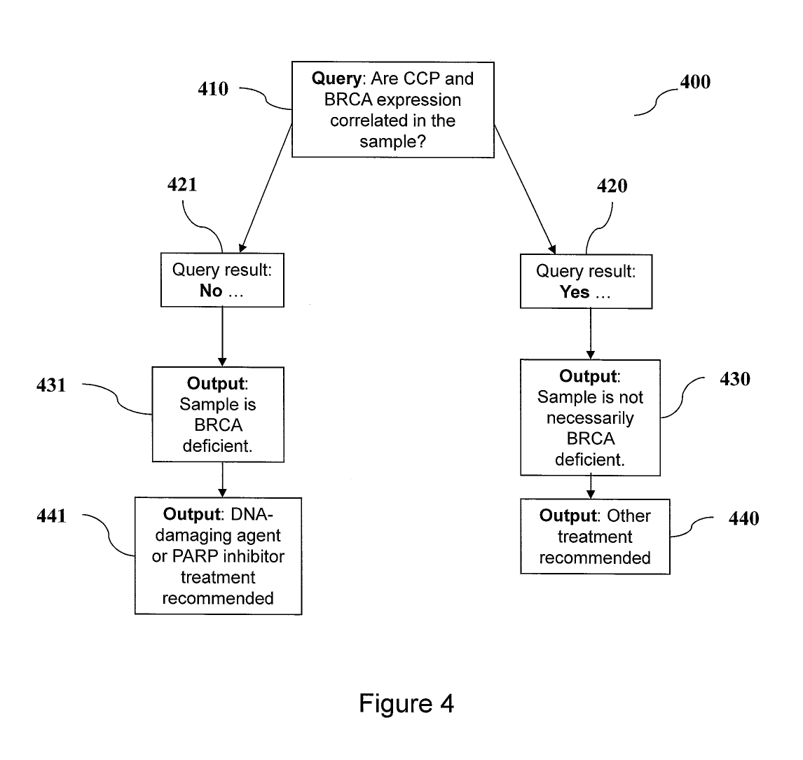

FIG. 4 illustrates one embodiment of a computer-implemented method [400] of the invention that may be implemented with the computer system [300] of the invention. The method [400] begins with a query ([410]), either sequentially or substantially simultaneously. If the answer to/result for this query is “Yes” [420], the method concludes [430] that the sample is BRCA deficient. If the answer to/result for this query is “No” [421], the method concludes [431] that the sample is not necessarily BRCA deficient. The method [400] may then proceed with more queries, make a particular treatment recommendation ([440], [441]), or simply end.

In some embodiments, the computer-implemented method of the invention [400] is open-ended. In other words, the apparent first step [410] in FIG. 4 may actually form part of a larger process and, within this larger process, need not be the first step/query. Additional steps may also be added onto the core methods discussed above. These additional steps include, but are not limited to, informing a health care professional (or the patient itself) of the conclusion reached; combining the conclusion reached by the illustrated method [400] with other facts or conclusions to reach some additional or refined conclusion regarding the patient's diagnosis, prognosis, treatment, etc.; making a recommendation for treatment; additional queries about additional biomarkers, clinical parameters, or other useful patient information (e.g., age at diagnosis, general patient health, etc.).

Regarding the above computer-implemented method [400], the answers to queries may be determined by the method instituting a search of patient data for the answer. For example, to answer the query [410], patient data may be searched for BRCA and CCP expression data. If such a comparison has not already been performed, the method may compare these data to some reference in order to determine if the respective expressions are high, low, average, etc. The method may also compare the respective expressions to determine if BRCA and CCP expression are correlated. Additionally or alternatively, the method may present one or more of the queries (e.g., [410]) to a user (e.g., a physician) of the computer system [300]. For example, the query [410] may be presented via an output module [324]. The user may then answer “Yes” or “No” via an input module [330]. The method may then proceed based upon the answer received. Likewise, the conclusions [430, 431, 440, 441] may be presented to a user of the computer-implemented method via an output module [324].

As used herein in the context of computer-implemented embodiments of the invention, “displaying” means communicating any information by any sensory means. Examples include, but are not limited to, visual displays, e.g., on a computer screen or on a sheet of paper printed at the command of the computer, and auditory displays, e.g., computer generated or recorded auditory expression of a patient sample's BRCA status.

The practice of the present invention may also employ conventional biology methods, software and systems. Computer software products of the invention typically include computer readable media having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. Basic computational biology methods are described in, for example, Setubal et al., INTRODUCTION TO COMPUTATIONAL BIOLOGY METHODS (PWS Publishing Company, Boston, 1997); Salzberg et al. (Ed.), COMPUTATIONAL METHODS IN MOLECULAR BIOLOGY, (Elsevier, Amsterdam, 1998); Rashidi & Buehler, BIOINFORMATICS BASICS: APPLICATION IN BIOLOGICAL SCIENCE AND MEDICINE (CRC Press, London, 2000); and Ouelette & Bzevanis, BIOINFORMATICS: A PRACTICAL GUIDE FOR ANALYSIS OF GENE AND PROTEINS (Wiley & Sons, Inc., 2^{nd} ed., 2001); see also, U.S. Pat. No. 6,420,108.

The present invention may also make use of various computer program products and software for a variety of purposes, such as probe design, management of data, analysis, and instrument operation. See U.S. Pat. Nos. 5,593,839; 5,795,716; 5,733,729; 5,974,164; 6,066,454; 6,090,555; 6,185,561; 6,188,783; 6,223,127; 6,229,911 and 6,308,170. Additionally, the present invention may have embodiments that include methods for providing genetic information over networks such as the Internet as shown in U.S. Ser. Nos. 10/197,621 (U.S. Pub. No. 20030097222); 10/063,559 (U.S. Pub. No. 20020183936), 10/065,856 (U.S. Pub. No. 20030100995); 10/065,868 (U.S. Pub. No. 20030120432); 10/423,403 (U.S. Pub. No. 20040049354).

In one aspect, the present invention provides methods of treating a cancer patient comprising determining whether BRCA and CCP expression are correlated in a sample from the patient and (1) recommending, prescribing, or administering a particular treatment regimen if BRCA and CCP expression are anti-correlated in the sample or (2) recommending, prescribing, or administering a particular treatment regimen if BRCA and CCP expression are correlated in the sample. In some embodiments, the particular treatment regimen comprises a DNA-damaging agent (e.g., platinum) chemotherapy if BRCA and CCP expression are anti-correlated in the sample. In some embodiments, the particular treatment regimen comprises PARP-inhibitor drugs if BRCA and CCP expression are anti-correlated in the sample. In some embodiments, if BRCA and CCP expression are correlated in the sample the particular treatment regimen comprises a regimen chosen from the group consisting of AC, FEC, FAC, FEC-T, Epirubicin-CMF, TAC, AC-Paclitaxel, AT, TC, T-Carboplatin, Lapatinib, Trastuzumab, Bevacizumab, Sunitinib, Docetaxel, Paclitaxel, Nano Paclitaxel, Docetaxel/capecitabine, Paclitaxel/gemcitabine, Docetaxel/gemcitabine, Gemcitabine, Trastuzumab/Docetaxel, Trastuzumab/Paclitaxel, Capecitabine, Lapatinib/Capecitabine, Ixabepilone, and Toco-P.

The methods of the invention are useful, inter alia, in identifying individuals who may benefit from germline BRCA testing but who may not meet the commonly applied criteria for identifying such individuals. For instance, commonly used criteria include personal history of cancer and significant family history of cancer. As used herein, “personal history of cancer” has its conventional meaning in the art (e.g., a previous cancer in the individual in question). As used herein, “significant family history of cancer” also has its conventional meaning in the art. Various guidelines have been devised and are used by healthcare professionals to determine whether an individual has a “significant family history of cancer.” These include guidelines of American Gastroenterological Association; American Society of Breast Surgeons; American Society of Clinical Oncology; American Society of Colon & Rectal Surgeons; Oncology Nursing Society; Society of Gynecologic Oncologists (e.g., women with breast cancer at ≦40 years, women with bilateral breast cancer (particularly if the first cancer was at ≦50 years); women with breast cancer at ≦50 years and a close relative† with breast cancer at ≦50 years; women of Ashkenazi Jewish ancestry with breast cancer at ≦50 years; women with breast or ovarian cancer at any age and two or more close relatives with breast cancer at any age (particularly if at least one breast cancer was at ≦50 years); unaffected women with a first or second degree relative that meets one of the above criteria), etc. Other widely accepted criteria include individuals with a personal or family history of breast cancer before age 50 or ovarian cancer at any age; individuals with two or more primary diagnoses of breast and/or ovarian cancer; individuals of Ashkenazi Jewish descent with a personal or family history of breast cancer before age 50 or ovarian cancer at any age; male breast cancer patients. A patient lacks a “significant family history of cancer” when one or more of these criteria are not met (usually all). Thus in some embodiments the patient to be assessed by the methods of the invention has a significant family history of cancer. In some embodiments the patient has a personal history of cancer.

In another aspect of the present invention, a kit is provided for practicing the methods and for use in the systems of the present invention. The kit may include a carrier for the various components of the kit. The carrier can be a container or support, in the form of, e.g., bag, box, tube, rack, and is optionally compartmentalized. The carrier may define an enclosed confinement for safety purposes during shipment and storage.

The kit includes various components useful in determining the expression of BRCA1 and/or BRCA2, the expression of at least two CCP genes, and optionally the expression of one or more housekeeping gene markers and/or the methylation status of BRCA1 and/or BRCA2. For example, the kit many include oligonucleotides specifically hybridizing under high stringency to mRNA or cDNA of BRCA1, BRCA2, or the genes in Tables 1 to 5 or Panels A to F. Such oligonucleotides can be used as PCR primers in RT-PCR reactions, or hybridization probes. In some embodiments the kit comprises reagents (e.g., probes, primers, and or antibodies) for determining the expression level of a panel of genes, where said panel comprises at least 25%, 30%, 40%, 50%, 60%, 75%, 80%, 90%, 95%, 99%, or 100% CCP genes (e.g., CCP genes in Tables 1 to 5 or Panels A to F). In some embodiments the kit consists of reagents (e.g., probes, primers, and or antibodies) for determining the expression level of no more than 2500 genes, wherein at least 5, 10, 15, 20, 30, 40, 50, 60, 70, 80, 90, 100, 120, 150, 200, 250, or more of these genes are CCP genes (e.g., Tables 1 to 5 or Panels A to F).

The oligonucleotides in the detection kit can be labeled with any suitable detection marker including but not limited to, radioactive isotopes, fluorephores, biotin, enzymes (e.g., alkaline phosphatase), enzyme substrates, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977). Alternatively, the oligonucleotides included in the kit are not labeled, and instead, one or more markers are provided in the kit so that users may label the oligonucleotides at the time of use.

Various other components useful in the detection techniques may also be included in the detection kit of this invention. Examples of such components include, but are not limited to, Taq polymerase, deoxyribonucleotides, dideoxyribonucleotides, other primers suitable for the amplification of a target DNA sequence, RNase A, and the like. In addition, the detection kit preferably includes instructions on using the kit for practice the prognosis method of the present invention using human samples.

### Example 1

The following example illustrates the validation of a CCP gene panel in predicting predicting time to chemical recurrence after radical prostatectomy in prostate cancer patients. The following CCP gene panel was tested:

TABLE 12
31-CCP Gene Cancer Recurrence
Signature

| | | |
|---|---|---|
| AURKA | DTL | PTTG1 |
| BUB1 | FOXM1 | RRM2 |
| CCNB1 | HMMR | TIMELESS |
| CCNB2 | KIF23 | TPX2 |
| CDC2 | KPNA2 | TRIP13 |
| CDC20 | MAD2L1 | TTK |
| CDC45L | MELK | UBE2C |
| CDCA8 | MYBL2 | UBE2S |
| CENPA | NUSAP1 | ZWINT |
| CKS2 | PBK | |
| DLG7 | PRC1 | |

Mean mRNA expression for the above 31 CCP genes was tested on 440 prostate tumor FFPE samples using a Cox Proportional Hazard model in Splus 7.1 (Insightful, Inc., Seattle Wash.). The p-value for the likelihood ratio test was 3.98×10⁻⁵. The mean of CCP expression is robust to measurement error and individual variation between genes.

The study further aimed at determining the optimal number of CCP genes to include in a CCP panel. As mentioned above, CCP expression levels are correlated to each other so it was possible that measuring a small number of genes would be sufficient, e.g., to predict prostate cancer outcome. In order to determine the optimal number of CCP genes for the signature, the predictive power of the mean was tested for randomly selected sets of from 1 to 30 of the CCP genes listed above. To evaluate how smaller subsets of the larger CCP set (i.e., smaller CCP panels) performed, the study also compared how well the signature predicted outcome as a function of the number of CCP genes included in the signature (FIG. 1). Time to chemical recurrence after prostate surgery was regressed on the CCP mean adjusted by the post-RP nomogram score. Data consist of TLDA assays expressed as deltaCT for 199 FFPE prostate tumor samples and 26 CCP genes and were analyzed by a CoxPH multivariate model. P-values are for the likelihood ratio test of the full model (nomogram+cell cycle mean including interaction) vs the reduced model (nomogram only). As shown in Table 13 below and FIG. 1, small CCP signatures (e.g., 2, 3, 4, 5, 6 CCP genes, etc.) add significantly to the Kattan-Stephenson nomogram:

TABLE 13

| # of CCP | Mean of log10 |
|---|---|
| genes | (p-value)* |
| 1 | −3.579 |
| 2 | −4.279 |
| 3 | −5.049 |
| 4 | −5.473 |
| 5 | −5.877 |
| 6 | −6.228 |

*For 1000 randomly drawn subsets, size 1 through 6, of cell cycle genes.

This simulation showed that there is a threshold range of CCP genes in a panel that provides significantly improved predictive power (FIG. 1).

### Example 2

### Patient Characteristics

Unselected human ovarian cancer tissues (235) were obtained under Institutional Review Board (IRB)-approved protocols. Table 9 shows the patient/cancer characteristics.

RNA/DNA Extraction from Frozen Cancers

10 μm thick sections from frozen cancer blocks in Tissue-Tek OCT (Qiagen, Valencia, Calif.) were homogenized using a TissueRuptor (Qiagen) after adding QIAzol lysis reagent, followed by RNA isolation using a QIAgen miRNAeasy Mini Kit per manufacturers protocol. A QIAamp DNA Mini Kit (QIAgen) was used to isolate DNA per the manufacturer's protocol with overnight incubation at 56° C. and RNaseA treatment.

### Quantitative-PCR—BRCA1

Reverse transcription was performed using a High-Capacity cDNA Reverse Transcription Kit (Applied Biosystems, Inc.) per manufacturer instructions. For pre-amplification, a 0.2× probe mix was made by combining 1 μL of 91 20× gene expression assays from Applied Biosystems Inc. and 9 μL of low-EDTA TE. Pre-amplification was performed using 2.54, of 2× TaqMan° PreAmp Master Mix (Applied Biosystems, Inc), 1.25 μL of 0.2× probe mix, and 1.25 μL cDNA. Applied Biosystems TaqMan assays (BRCA 1: Hs00173233_ml/Hs00173237_ml/Hs01556190_ml/Hs01556191_ml; BRCA2: Hs00609060_ml; housekeepers: Hs99999908_ml (GUSB)/Hs00188166_ml (SDHA)/Hs00237047_ml (YWHAZ)/Hs00824723_ml (UBC)/Hs00609297_ml (HMBS)) were used for pre-amplification and qPCR on a Fluidigm (South San Francisco, Calif.) BioMark instrument. Cycle conditions were 95° C. for 10 minutes, 17 cycles of 95° C. for 15 seconds and 60° C. for 4 minutes. The PCR products were diluted 1:5 with low-EDTA TE. Samples were assessed on gene expression M48 dynamic arrays (Fluidigm) per manufacturer's protocol.

### Quantitative PCR—CCP Score

500 ng-1 μg of RNA was treated with Amplification Grade Deoxyribonuclease I (Sigma-Aldrich Inc.) in a 10 μL reaction at room temperature for 30 minutes. 1 μL of Stop Solution is then added and heated to 70° C. for 10 minutes. 14 μLs of RNase-free water is added to make 1 ug of RNA in 25 μLs to be used in a 50 μL reverse transcription reaction using High-Capacity cDNA Reverse Transcription Kit (Applied Biosystems, Inc.)

Pre-Amplification was done using a 0.2× probe mix made combining 1 μL of the 48 individual 20× gene expression assays from Applied Biosystems, Inc. and 52 μLs of low-EDTA TE. Pre-amplification was performed using 2.5 μLs of TaqMan® PreAmp Master Mix (2×) (Applied Biosystems, Inc.), 1.25 μLs of the 0.2× probe mix, and 1.25 μL cDNA.

The range of expression of the genes involved in the calculation of CCP score was too large to allow accurate quantification under uniform conditions. Two pre-amplifications were run independently at each of the two cycle conditions, 8 and 18 cycles. Cycle conditions were 95° C. for 10 minutes and 8/18 cycles of 95° C.×15 seconds and 60° C.×4 minutes. The products were then diluted 1:5 using low-EDTA TE. Samples were run versus the 48 assays (Table 10) on the Fluidigm Gene Expression 48.48 Dynamic Arrays per manufacturers' protocol.

qPCR Analysis

The comparative C_{T} method was used to calculate relative gene expression using the C_{T} for the BRCA2 assay, the average C_{T}s from the BRCA 1 assays, and the average C_{T}s from housekeeper genes. qPCR was performed in 220 cancers where high quality RNA was obtained.

### BRCA1 Methylation Assay

MeAH-011E Methyl-ProfilerTM DNA Methylation PCR Assay Human Breast Cancer, Signature Panel (24-Genes, 385-Well Plates) was used per manufacturers' protocol for the 4-sample format. 125 ng RNase treated genomic DNA was used per restriction enzyme digestion, for a total of 500 ng. Incubation of digestion reactions was performed at 37° C. for 6 hours.

### Data Analysis

### Calculation of CCP Score

CCP scores were calculated for each sample in the following manner. C_{T} values less than 8 were considered to be above the limit of detection and were removed from the analysis. Data from the two pre-amplification cycling conditions were normalized by subtracting off the average of the C_{T} values of the genes that were not missing any values and whose C_{T} were between 8 and 23 under both conditions. These centered C_{T} values were averaged for each gene with at least two C_{T} values whose standard deviation was less than or equal to 3. ΔC_{T} was calculated as the difference in centered C_{T} values between the gene of interest and the average of the housekeeper genes. ΔC_{T} was then centered for each gene by the average ΔC_{T} on all the samples that were not missing ΔC_{T} for any gene. The negative of the average of the centered ΔC_{T} across the cell-cycle genes is the CCP score.

### Abnormal BRCA1 Expression

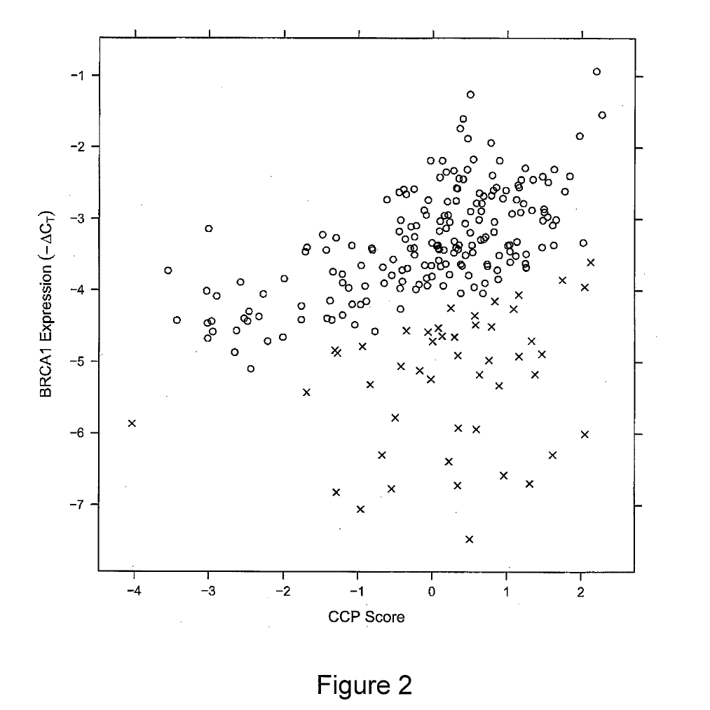

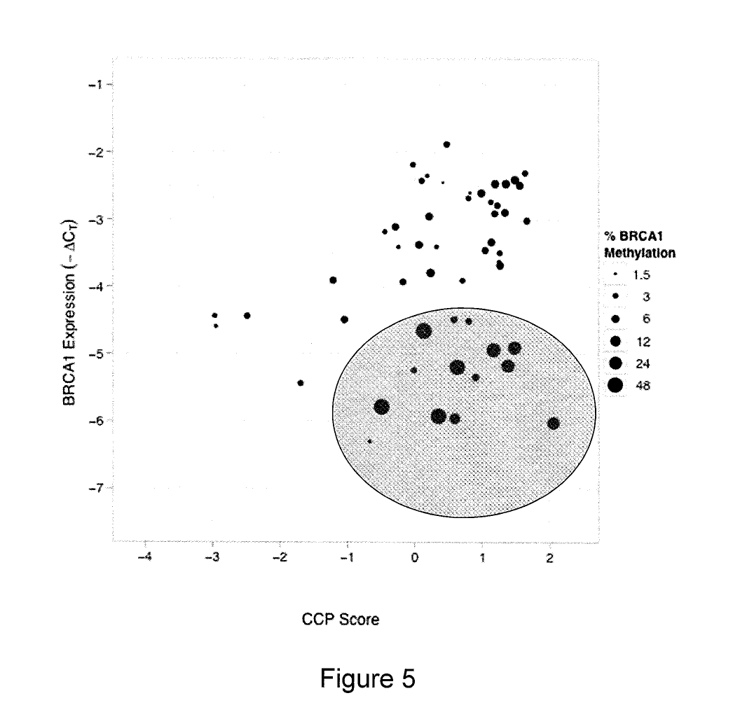

FIG. 2 shows the relationship between BRCA1 and cell-cycle gene (as measured by the CCP score) expression. The samples where BRCA1 and cell-cycle gene expression are correlated (circles, correlation=0.65) are considered to have normal expression. The samples with high CCP scores but low expression of BRCA1 are considered to have abnormal expression (i.e., anti-correlation; X's). FIG. 5 shows that, upon further analysis, the samples with anti-correlation between BRCA1 and CCP expression (those within the shaded circle) generally turned out to have BRCA1 hypermethylation (larger points indicate higher extent of methylation). An iterative method was used to identify these samples. First, a linear model was fit with BRCA1 expression as the response and CCP score as the only predictor. Next, the differences between the observed and fitted BRCA1 expression from the previous step were separated into two clusters using k-means clustering. Last, the lower cluster was removed and the process was repeated until the cluster membership did not change from one iteration to the next.

### BRCA Deficiency

A patient sample was considered BRCA deficient (79 out of 242 tested) if it had a mutation in BRCA1/2 (41 out of 227 tested), abnormal expression of BRCA1 (47/239), or more than 10% methylation of BRCA1 (9 out of 53 tested).

### Association Between PFS and BRCA Deficiency

The association between progression free survival (PFS) and BRCA deficiency was tested using the partial likelihood ratio test from a Cox's proportional hazards model with PFS as the response and BRCA deficiency as the only predictor. The hazard ratio (HR) for deficient patients versus non-deficient patients was 0.66 (p-value=0.014, n=193, 16% censoring), indicating decreased risk of disease progression in deficient patients.

TABLE 14

| Total Number of Patients | 235 | |
|---|---|---|
| Age at | Range | 23-92 |
| Diagnosis | Median | 60 |
| | Unknown | 20 (8.5%) |
| Follow-up Time | Range | 19-6141days |
| | Median | 1071 days |
| | Unknown | 8 (3.5%) |
| Stage | 1 | 11 (5%) |
| | 2 | 14 (6%) |
| | 3 | 156 (66%) |
| | 4 | 33 (14%) |
| | Unknown | 21 (9%) |
| Histology | Serous | 186 (79%) |
| | Non-serous | 13 (6%) |
| | Mixed | 13 (6%) |
| | Unknown | 22 (95) |
| Grade | 1 | 13 (5.5%) |
| | 2 | 19 (8%) |
| | 3 | 180 (76.5%) |
| | Unknown | 23 (10%) |
| Residual | 0 | 12 (5) |
| Disease after | ≦1 cm | 126 (53.5%) |
| Surgery | >1 cm | 60 (25.5%) |
| | Unknown | 37 (16%) |
| Surgery | Yes | 230 (98%) |
| | No | 5 (2%) |
| | Unknown | 0 |
| Chemotherapy | No chemotherapy | 9 (3.8%) |
| | Unknown | 33 (14%) |
| | Platinum (cis or | 17 (7.2%) |
| | carboplatin)-based | |
| | (no taxane) | |
| | Platinum plus | 176 (74.9%) |
| | taxane (paclitaxel | |
| | or docetaxel)-based | |

TABLE 15

| CCP | Entrez | Housekeeper | Entrez |
|---|---|---|---|
| Genes | GeneId | Genes | GeneId |
| ASF1B | 55723 | CLTC | 1213 |
| ASPM | 259266 | MMADHC | 27249 |
| BIRC5 | 332 | MRFAP1 | 93621 |
| BUB1B | 701 | PPP2CA | 5515 |
| C18orf24 | 220134 | PSMA1 | 5682 |
| CDC20 | 983 | PSMC1 | 5700 |
| CDC2 | 991 | RPL13A | 23521 |
| CDCA3 | 83461 | RPL37 | 6167 |
| CDCA8 | 55143 | RPL38 | 6169 |
| CDKN3 | 1033 | RPL4 | 6124 |
| CENPF | 1063 | RPL8 | 6132 |
| CENPM | 79019 | RPS29 | 6235 |
| CEP55 | 55165 | SLC25A3 | 5250 |
| DLGAP5 | 9787 | TXNL1 | 9352 |
| DTL | 51514 | UBA52 | 7311 |
| FOXM1 | 2305 | | |
| KIAA0101 | 9768 | | |
| KIF11 | 3832 | | |
| KIF20A | 10112 | | |
| MCM10 | 55388 | | |
| NUSAP1 | 51203 | | |
| ORC6L | 23594 | | |
| PBK | 55872 | | |
| PLK1 | 5347 | | |
| PRC1 | 9055 | | |
| PTTG1 | 9232 | | |
| RAD51 | 5888 | | |
| RAD54L | 8438 | | |
| RRM2 | 6241 | | |
| TK1 | 7083 | | |
| TOP2A | 7153 | | |

### Example 3

### Description of Clinical Data

The samples in this study consisted of 216 fresh frozen breast tumors from 4 commercial sources. All but one had ER, PR, and HER2 status. Unless stated otherwise, all assay and statistical details for this study were as described in Example 2 above.

### ER/PR/HER2 Subtype Classification

Three ER-patients were PR+. As such, each sample was assigned one of three subtypes based on ER status first and then on HER2 status in the ER-tumors: 113 ER+, 64 triple negative, and 38 ER−/HER2+. One ER− patient was missing HER2 status. As a result her tumor subtype could not be assigned.

### BRCA1 Expression

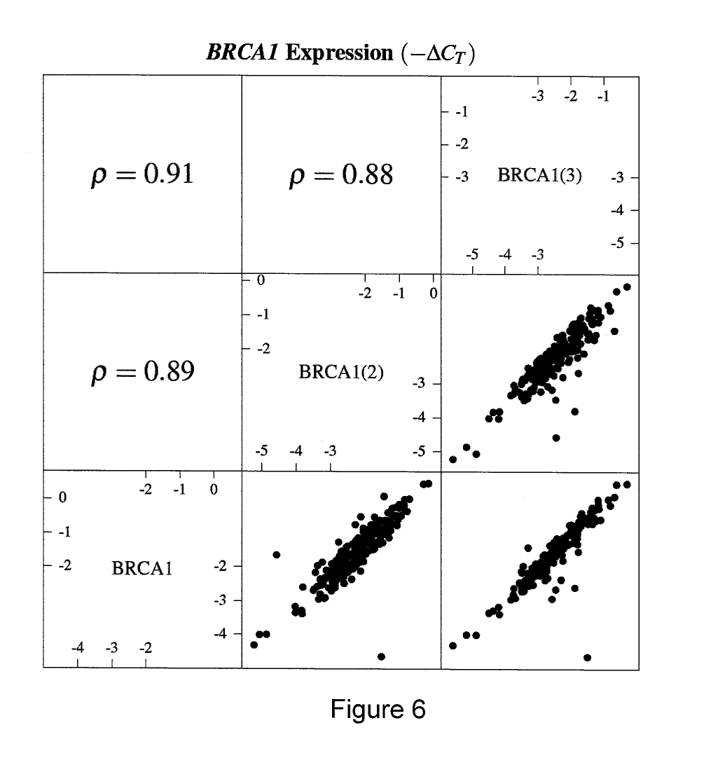

BRCA1 expression was measured and calculated for 215 patients' tumors. Three qPCR assays for BRCA1 (Hs00173233_ml (BRCA1), Hs00173237_ml (BRCA1(2)), and Hs01556190_ml (BRCA1(3))) and three housekeeper genes (MMADHC, RPS23, and SDHA) were used to measure BRCA1 expression on these samples. Each sample was preamplified with all the assays 4 times: twice for 12 cycles and twice for 18 cycles. C_{T} was determined for each assay-sample-preamp. For each sample, the genes with C_{T} between 8 and 23 on all preamps were identified as centering genes. They were averaged for each preamp. This quantity was subtracted from the C_{T} of each measurement to put the C_{T} from different numbers of cycles of preamp on the same scale. All replicates with C_{T} greater than 8 were averaged for each assay. ΔC_{T} was calculated for each BRCA1 assay by subtracting the average of the three housekeeper genes. The pairwise relationships between the normalized expression for the BRCA1 assays are shown in FIG. 6.

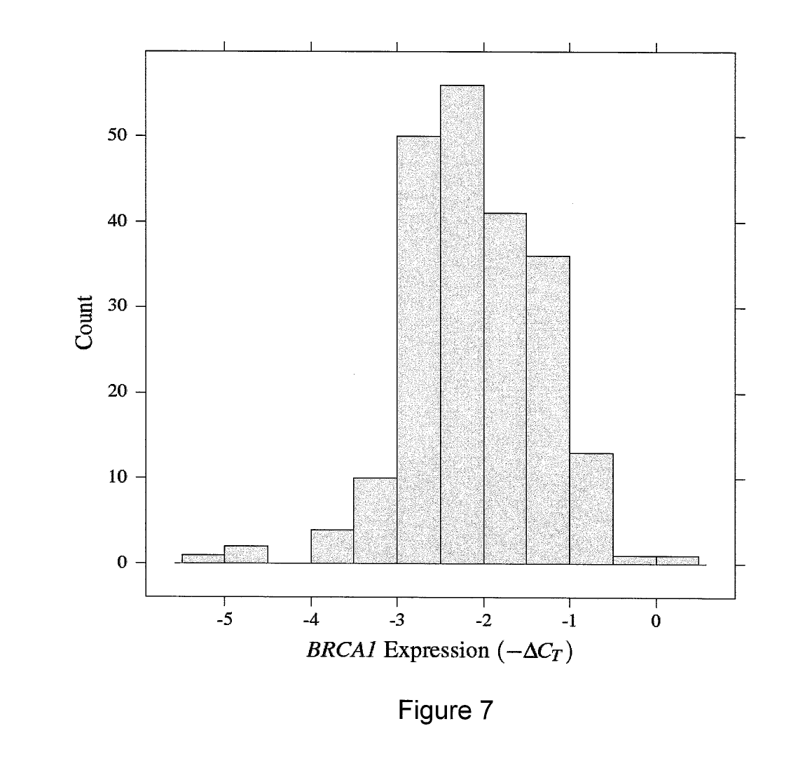

As the correlation of the three BRCA1 assays was high, BRCA1 expression was calculated as the average −ΔC_{T} of the three assays. FIG. 7 is a histogram of the final BRCA1 expression values.

### CCP Score

Cell-cycle gene expression was measured and calculated for 215 patients' samples in the same manner as BRCA1 expression, with a few exceptions. First, the ProAssay04 set of assays, which consists of 31 cell-cycle genes and 15 housekeepers (Table 15 above), was used instead of 3 housekeepers and 3 assays for the gene of interest. Second, 8 and 18 cycles of preamp were used instead of 12 and 18. Lastly, before averaging all the genes, each gene was centered by the average expression of that gene in the samples where all the cell-cycle genes performed well.

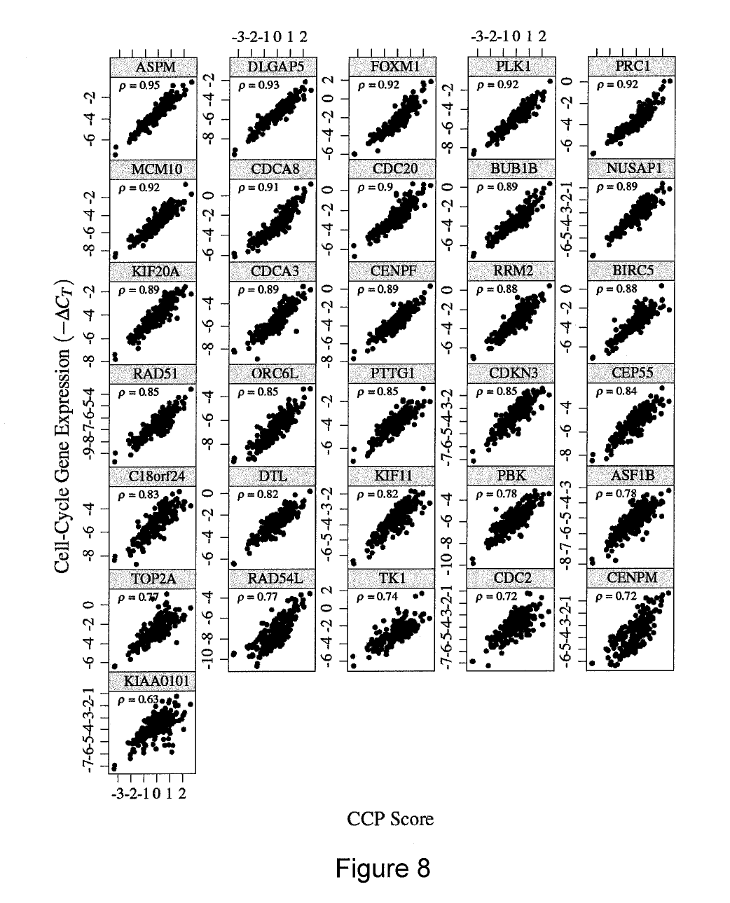

The correlation between each of the cell-cycle genes and the CCP score is shown in FIG. 8.

### Abnormal BRCA1 Expression

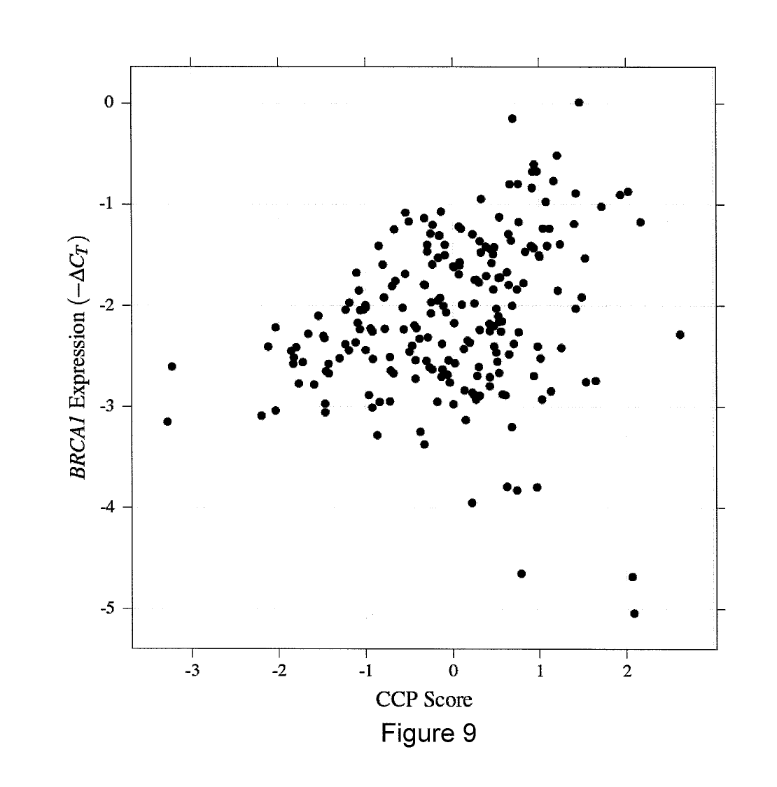

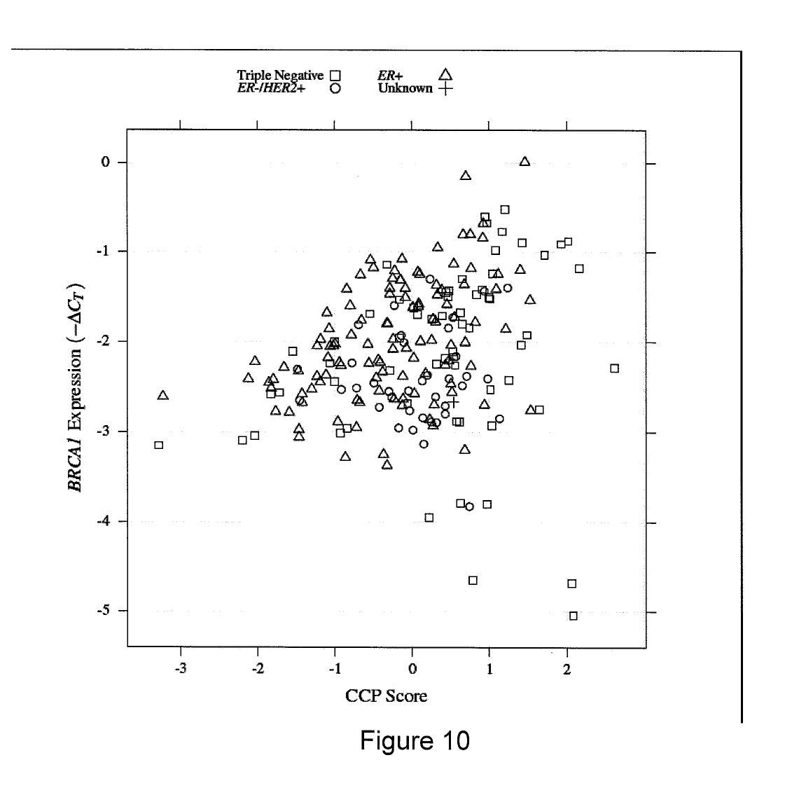

FIG. 9 is a plot of CCP score and BRCA1 expression. FIG. 10 is a plot of CCP score and BRCA1 expression colored by ER/PR/HER2 subtype as determined by IHC.

### BRCA1 Methylation

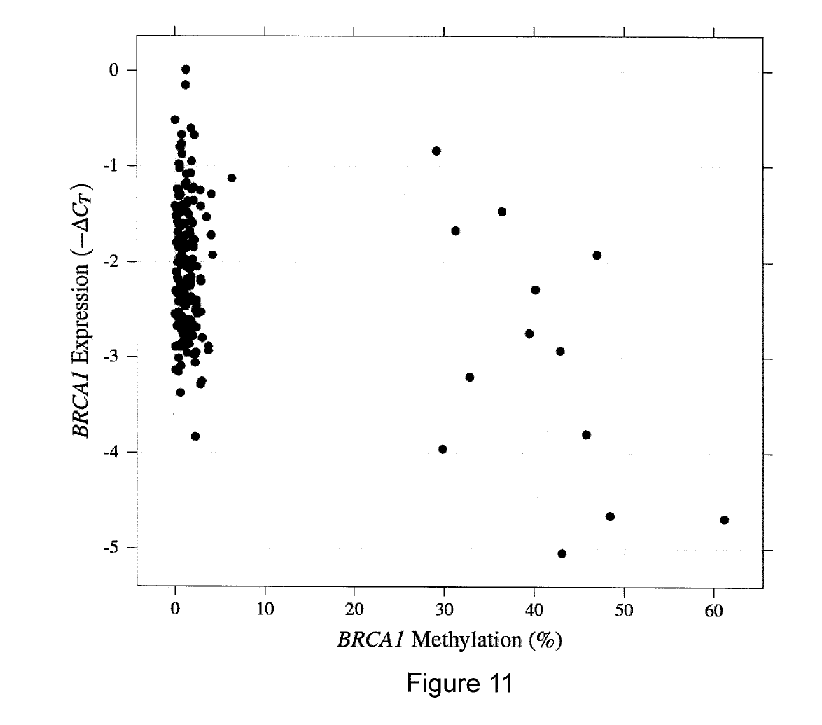

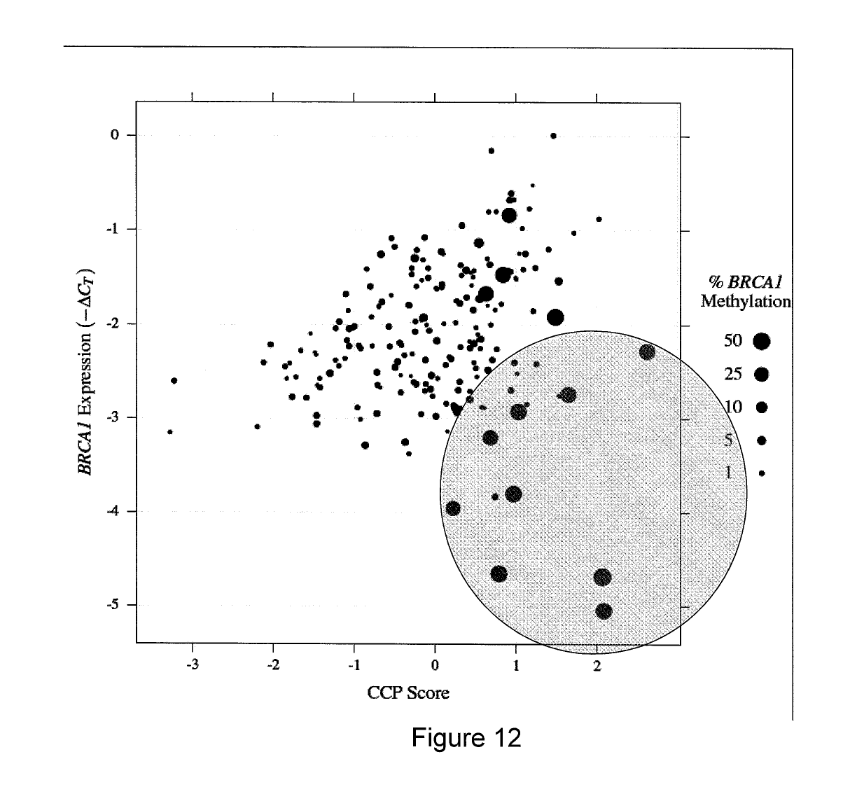

Methylation of the BRCA1 promoter region was measured in 199 tumors. FIG. 11 shows the relationship between BRCA1 methylation and expression. FIG. 12 shows the relationship between BRCA1 expression, CCP score, and BRCA1 methylation. A distinct subset of samples with anti-correlated CCP and BRCA1 expression can be seen in the lower right quadrant of FIG. 7 (shaded circle). Most of these samples show high CCP expression paired with average to low BRCA1 expression. It is further notable that such samples generally showed hypermethylation.

It is specifically contemplated that any embodiment of any method or composition of the invention may be used with respect to any other method or composition of the invention.

In the context of genes and gene products, the name of the gene is generally italicized herein following convention. In such cases, the italicized gene name is generally to be understood to refer to the gene (i.e., genomic), its mRNA (or cDNA) product, and/or its protein product. Generally, though not always, a non-italicized gene name refers to the gene's protein product.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

Unless otherwise defined, all technical and scientific terms used herein have the same meaning as commonly understood by one of ordinary skill in the art to which this invention pertains. In case of conflict, the present specification, including definitions, will control. In addition, the materials, methods, and examples are illustrative only and not intended to be limiting.

Other features and advantages of the invention will be apparent from the preceding detailed description and from the following claims

1-3. (canceled)

4. A method for detecting BRCA1 deficiency in a sample from a patient comprising
(1) measuring a plurality of genes in said sample, wherein said plurality of genes consists of at most 2,000 genes and comprises BRCA1 and at least three test genes chosen from the group consisting of ASF1B, ASPM, BIRC5, BUB1B, C18 orf24, CDC20, CDC2, CDCA3, CDCA8, CDKN3, CENPF, CENPM, CEP55, DLGAP5, DTL, FOXM1, KIAA0101, KIF11, KIF20A, MCM10, NUSAP1, ORC6L, PBK, PLK1, PRC1, PTTG1, RAD51, RAD54L, RRM2, TK1, and TOP2A;
(2) determining whether BRCA1 expression is correlated to the overall expression of said at least three test genes in said sample; and
(3) diagnosing said sample as comprising BRCA-deficient cells based at least in part on detection of an anti-correlation in said sample between BRCA1 expression and the overall expression of said at least three test genes.

5-6. (canceled)

7. The method of claim 5, further comprising diagnosing said sample as comprising cells with BRCA hypermethylation based at least in part on detection of an anti-correlation in said sample between BRCA1 expression and the overall expression of said at least three test genes.

8. A method of diagnosing a patient's likelihood of progression-free survival comprising:
measuring expression of a plurality of genes in a sample from said patient, wherein said plurality of genes consists of at most 2,000 genes and comprises BRCA1 and at least three test genes chosen from the group consisting of ASF1B, ASPM, BIRC5, BUB1B, C18orf24, CDC20, CDC2, CDCA3, CDCA8, CDKN3, CENPF, CENPM, CEP55, DLGAP5, DTL, FOXM1, KIAA0101, KIF11, KIF20A, MCM10, NUSAP1, ORC6L, PBK, PLK1, PRO, PTTG1, RAD51, RAD54L, RRM2, TK1, and TOP2A;
determining whether there is an anti-correlation in said sample between BRCA1 expression and expression of said at least three test genes; and
diagnosing said patient as having (a) an increased likelihood of longer progression-free survival based at least in part on detecting an anti-correlation in said sample between BRCA1 expression and expression of said at least three test genes or (b) no increased likelihood of longer progression-free survival based at least in part on not detecting an anti-correlation in said sample between BRCA1 expression and expression of said at least three test genes.

9. A method of predicting a patient's response to a treatment regimen comprising either DNA-damaging agents or PARP pathway inhibitors, the method comprising:
measuring expression of a plurality of genes in a sample from a patient, wherein said plurality of genes consists of at most 2,000 genes and comprises BRCA1 and at least three test genes chosen from the group consisting of ASF1B, ASPM, BIRC5, BUB1B, C18 orf24, CDC20, CDC2, CDCA3, CDCA8, CDKN3, CENPF, CENPM, CEP55, DLGAP5, DTL, FOXM1, KIAA0101, KIF11, KIF20A, MCM10, NUSAP1, ORC6L, PBK, PLK1, PRC1, PTTG1, RAD51, RAD54L, RRM2, TK1, and TOP2A;
determining whether there is an anti-correlation in said sample between BRCA1 expression and expression of said at least three test genes; and
diagnosing said patient as having (a) an increased likelihood of response to said treatment based at least in part on detecting an anti-correlation in said sample between BRCA1 expression and expression of said at least three test genes or (b) no increased likelihood of response to said treatment based at least in part on not detecting an anti-correlation in said sample between BRCA1 expression and expression of said at least three test genes.

10. (canceled)

11. A system for determining gene expression in a tumor sample, comprising:
(1) a sample analyzer for measuring expression of a plurality of genes in a sample from a patient, wherein said plurality of genes consists of at most 2,000 genes and comprises the test genes BRCA1 and at least three genes selected from the group consisting of ASF1B, ASPM, BIRC5, BUB1B, C18 orf24, CDC20, CDC2, CDCA3, CDCA8, CDKN3, CENPF, CENPM, CEP55, DLGAP5, DTL, FOXM1, KIAA0101, KIF11, KIF20A, MCM10, NUSAP1, ORC6L, PBK, PLK1, PRC1, PTTG1, RAD51, RAD54L, RRM2, TK1, and TOP2A, and wherein the sample analyzer contains the sample, mRNA from the sample and expressed from the plurality of genes, or cDNA synthesized from said mRNA;
(2) a first computer program for (a) receiving gene expression data on at least each of said test genes, (b) weighting the determined expression of at least each of said test genes with a predefined coefficient, and (c) combining the weighted expression to provide a CCP test value representing the expression level of ASF1B, ASPM, BIRC5, BUB1B, C18orf24, CDC20, CDC2, CDCA3, CDCA8, CDKN3, CENPF, CENPM, CEP55, DLGAP5, DTL, FOXM1, KIAA0101, KIF11, KIF20A, MCM10, NUSAP1, ORC6L, PBK, PLK1, PRC1, PTTG1, RAD51, RAD54L, RRM2, TK1, and TOP2A;
(3) a second computer program for comparing the expression of BRCA1 to the CCP test value, wherein said second computer program (a) correlates high expression of BRCA1 coupled with a high CCP test value to correlation between BRCA1 and CCP expression; (b) correlates an absence of high BRCA1 expression coupled with a low CCP test value to correlation between BRCA1 and CCP expression; (c) correlates high expression of BRCA1 coupled with a low CCP test value to anti-correlation between BRCA1 and CCP expression; and (d) correlates an absence of high BRCA1 expression coupled with a high CCP test value to anti-correlation between BRCA1 and CCP expression.

12-22. (canceled)

24. The system of claim 11, further comprising a third computer program that concludes that the sample is BRCA deficient if BRCA expression and CCP expression are anti-correlated in the sample.

25-26. (canceled)

27. The method of claim 4, wherein anti-correlation between BRCA1 expression and expression of said at least three test genes is found when the sample shows an absence of high BRCA1 expression coupled with high overall expression of ASF1B, ASPM, BIRC5, BUB1B, C18orf24, CDC20, CDC2, CDCA3, CDCA8, CDKN3, CENPF, CENPM, CEP55, DLGAP5, DTL, FOXM1, KIAA0101, KIF11, KIF20A, MCM10, NUSAP1, ORC6L, PBK, PLK1, PRC1, PTTG1, RAD51, RAD54L, RRM2, TK1, and TOP2A.

28. The method of claim 8, wherein anti-correlation between BRCA1 expression and expression of said at least three test genes is found when the sample shows an absence of high BRCA1 expression coupled with high overall expression of ASF1B, ASPM, BIRC5, BUB1B, C18orf24, CDC20, CDC2, CDCA3, CDCA8, CDKN3, CENPF, CENPM, CEP55, DLGAP5, DTL, FOXM1, KIAA0101, KIF11, KIF20A, MCM10, NUSAP1, ORC6L, PBK, PLK1, PRC1, PTTG1, RAD51, RAD54L, RRM2, TK1, and TOP2A.

29. The method of claim 9, wherein anti-correlation between BRCA1 expression and expression of said at least three test genes is found when the sample shows an absence of high BRCA1 expression coupled with high overall expression of ASF1B, ASPM, BIRC5, BUB1B, C18orf24, CDC20, CDC2, CDCA3, CDCA8, CDKN3, CENPF, CENPM, CEP55, DLGAP5, DTL, FOXM1, KIAA0101, KIF11, KIF20A, MCM10, NUSAP1, ORC6L, PBK, PLK1, PRC1, PTTG1, RAD51, RAD54L, RRM2, TK1, and TOP2A.
